SEQUENCE LISTING (87 sequences)

SEQ ID NO: 1 - protein, 20 aa, Mycobacterium tuberculosis
EMWDIRNRGVIPAGALPRVR

SEQ ID NO: 2 - protein, 30 aa, Mycobacterium tuberculosis
AKFRSVRVVVITGSVTAAPVRVSETLRRLI

SEQ ID NO: 3 - protein, 13 aa, Mycobacterium tuberculosis
ESVRLAFVAALQH

SEQ ID NO: 4 - protein, 14 aa, Mycobacterium tuberculosis
GLLITIRSPRSGIA

SEQ ID NO: 5 - protein, 13 aa, Mycobacterium tuberculosis
AQLLWQLPLLSIG

SEQ ID NO: 6 - protein, 27 aa, Mycobacterium tuberculosis
ADLVRELVTILPIVLVIAAVAAYLLSR

SEQ ID NO: 7 - protein, 20 aa, Mycobacterium tuberculosis
AAYLLSRAALRPVDRIRAAA

SEQ ID NO: 8 - protein, 20 aa, Mycobacterium tuberculosis
TTLNTMLTRLQRALAHEQQF

SEQ ID NO: 9 - protein, 21 aa, Mycobacterium tuberculosis
DLFVSIDPDHLRRILTAVLDN

SEQ ID NO: 10 - protein, 17 aa, Mycobacterium tuberculosis
SGLGLAIVAALTTTHGG

SEQ ID NO: 11 - protein, 19 aa, Mycobacterium tuberculosis
GRLPRKGPWQQKVIKELPQ

SEQ ID NO: 12 - protein, 20 aa, Mycobacterium tuberculosis
GKIVVLMGAVGTMKPETQAA

SEQ ID NO: 13 - protein, 14 aa, Mycobacterium tuberculosis
GEIIFISGRLNGAA

SEQ ID NO: 14 - protein, 13 aa, Mycobacterium tuberculosis
ARVRSMSPLEIAD

SEQ ID NO: 15 - protein, 15 aa, Mycobacterium tuberculosis
EQILFRRLAPFVGGF

SEQ ID NO: 16 - protein, 14 aa, Mycobacterium tuberculosis
AALVRALTACGCSS

SEQ ID NO: 17 - protein, 21 aa, Mycobacterium tuberculosis
DKWTLCQILYWRGVGTCISGD

SEQ ID NO: 18 - protein, 17 aa, Mycobacterium tuberculosis
TKVLGLYTQAQVLAYCG

SEQ ID NO: 19 - protein, 18 aa, Mycobacterium tuberculosis
DQVTMHQVLMAQLALAGG

SEQ ID NO: 20 - protein, 22 aa, Mycobacterium tuberculosis
EGVRLLGAAAALRQQTRQVRFK

SEQ ID NO: 21 - protein, 13 aa, Mycobacterium tuberculosis
ELMARAAVLGSAH

SEQ ID NO: 22 - protein, 13 aa, Mycobacterium tuberculosis
AVIVRSELLTQYL

SEQ ID NO: 23 - protein, 20 aa, Mycobacterium tuberculosis
GTLRHLDPPVRRSGGREQHL

SEQ ID NO: 24 - protein, 13 aa, Mycobacterium tuberculosis
TAWITAVVPGLMV

SEQ ID NO: 25 - protein, 13 aa, Mycobacterium tuberculosis
APMVFSATKGMTA

SEQ ID NO: 26 - protein, 14 aa, Mycobacterium tuberculosis
TCAMRRLAHRFSGG

SEQ ID NO: 27 - protein, 13 aa, Mycobacterium tuberculosis
SVVRRKQTLLSAQ

SEQ ID NO: 28 - protein, 13 aa, Mycobacterium tuberculosis
GVVHRNPAVTVAE

SEQ ID NO: 29 - protein, 22 aa, Mycobacterium tuberculosis
PGVVATHAVRTLGTTGSRAIGL

SEQ ID NO: 30 - protein, 25 aa, Mycobacterium tuberculosis
PQWRRARVRLCGRWWRRSNTTRGAS

SEQ ID NO: 31 - protein, 18 aa, Mycobacterium tuberculosis
ARLMVGAVRRHRPGSLQR

SEQ ID NO: 32 - protein, 12 aa, Mycobacterium tuberculosis
AAMRNMKSTSHE

SEQ ID NO: 33 - protein, 14 aa, Mycobacterium tuberculosis
QTIVMLWTAAVGCA

SEQ ID NO: 34 - protein, 19 aa, Mycobacterium tuberculosis
LCMLMLGLLMLIFSEHRSS

SEQ ID NO: 35 - protein, 20 aa, Mycobacterium tuberculosis
SALVLVGLGLCGSGVALCLT

SEQ ID NO: 36 - protein, 16 aa, Mycobacterium tuberculosis
SELVRFELLAGVRESE

SEQ ID NO: 37 - protein, 14 aa, Mycobacterium tuberculosis
VDYLIAATAIVVDA

SEQ ID NO: 38 - protein, 15 aa, Mycobacterium tuberculosis
QGIEYYKSSVLPQIE

SEQ ID NO: 39 - protein, 16 aa, Mycobacterium tuberculosis
EGWIVYARSTTIQAQS

SEQ ID NO: 40 - protein, 10 aa, Mycobacterium tuberculosis
TRRMYSNYGF

SEQ ID NO: 41 - protein, 23 aa, Mycobacterium tuberculosis
FGYRVQESAFEAMLTKGQLAKLV

SEQ ID NO: 42 - protein, 26 aa, Mycobacterium tuberculosis
DNIRIYKIRGVAAVTFYGRGRLVSAE

SEQ ID NO: 43 - protein, 23 aa, Mycobacterium tuberculosis
RSYILRAGISSLFRYIEGVHGER

SEQ ID NO: 44 - protein, 20 aa, Mycobacterium tuberculosis
SAMRPQDRLLVGNWVDDSLL

SEQ ID NO: 45 - protein, 13 aa, Mycobacterium tuberculosis
LYLVGLEPYVQFE

SEQ ID NO: 46 - protein, 13 aa, Mycobacterium tuberculosis
AGFRILEARRFPI

SEQ ID NO: 47 - protein, 19 aa, Mycobacterium tuberculosis
IRYRARYVNGQLNMCLARI

SEQ ID NO: 48 - protein, 16 aa, Mycobacterium tuberculosis
ALLVAYLPARSREEMF

SEQ ID NO: 49 - protein, 13 aa, Mycobacterium tuberculosis
NRLRLAATHAVRT

SEQ ID NO: 50 - protein, 21 aa, Mycobacterium tuberculosis
APLQRRFRDAFTATAHFQVNE

SEQ ID NO: 51 - protein, 18 aa, Mycobacterium tuberculosis
SRELPGRVLLDQPADVSM

SEQ ID NO: 52 - protein, 18 aa, Mycobacterium tuberculosis
EPVVTVDVTAMSAVLEID

SEQ ID NO: 53 - protein, 23 aa, Mycobacterium tuberculosis
AVIQVSDRAVRGWRTGDIRPERY

SEQ ID NO: 54 - protein, 17 aa, Mycobacterium tuberculosis
PDLRGALLLAVTLGLVT

SEQ ID NO: 55 - protein, 16 aa, Mycobacterium tuberculosis
PDWGWLSVATVGSFLA

SEQ ID NO: 56 - protein, 21 aa, Mycobacterium tuberculosis
GAVLGVAVMVILIGKPEHGTA

SEQ ID NO: 57 - protein, 15 aa, Mycobacterium tuberculosis
AAICFIAVAVAAAVL

SEQ ID NO: 58 - protein, 15 aa, Mycobacterium tuberculosis
TKLVRLTKAQFDEIA

SEQ ID NO: 59 - protein, 14 aa, Mycobacterium tuberculosis
ADLVLAGPAASREH

SEQ ID NO: 60 - protein, 16 aa, Mycobacterium tuberculosis
YAYEYFIRHNPLSDYA

SEQ ID NO: 61 - protein, 18 aa, Mycobacterium tuberculosis
FPVRGLVRGRRTLTLLEA

SEQ ID NO: 62 - protein, 16 aa, Mycobacterium tuberculosis
EVLRILRRRSLAALRA

SEQ ID NO: 63 - protein, 18 aa, Mycobacterium tuberculosis
RVILHSPYGLRVHGPLAL

SEQ ID NO: 64 - protein, 15 aa, Mycobacterium tuberculosis
AAWVINGLANAYNDT

SEQ ID NO: 65 - protein, 27 aa, Mycobacterium tuberculosis
DQYRARNVAVMNAYVSWTRSALSDLPR

SEQ ID NO: 66 - protein, 25 aa, Mycobacterium tuberculosis
SDLLADAVERYLQWLSKSSSQLKHA

SEQ ID NO: 67 - protein, 13 aa, Mycobacterium tuberculosis
GPVVVHPSWWSAA

SEQ ID NO: 68 - protein, 16 aa, Mycobacterium tuberculosis
ITAVVLIDVPSTVAGA

SEQ ID NO: 69 - protein, 14 aa, Mycobacterium tuberculosis
AAVVRHGATTLQRP

SEQ ID NO: 70 - protein, 18 aa, Mycobacterium tuberculosis
RPVRRVLLFVVPSSGPAP

SEQ ID NO: 71 - protein, 24 aa, Mycobacterium tuberculosis
GSVRQLPSVLKPPLITLRTLTLSG

SEQ ID NO: 72 - protein, 18 aa, Mycobacterium tuberculosis
GAVLRLVVRFAEPLPPSP

SEQ ID NO: 73 - protein, 19 aa, Mycobacterium tuberculosis
AGYLLTYTIANNGKEFAEL

SEQ ID NO: 74 - protein, 18 aa, Mycobacterium tuberculosis
AAVRKRMVIGLSTGSDDD

SEQ ID NO: 75 - protein, 20 aa, Mycobacterium tuberculosis
ALLLRDVLQWKSAEVADAIG

SEQ ID NO: 76 - protein, 25 aa, Mycobacterium tuberculosis
NSLLQRARSQLQTVRPSAADRLSAA

SEQ ID NO: 77 - protein, 20 aa, Mycobacterium tuberculosis
MSWVMVSPELVVAAAADLAG

SEQ ID NO: 78 - protein, 17 aa, Mycobacterium tuberculosis
AAFYAQFVQALTSGGAY

SEQ ID NO: 79 - protein, 14 aa, Mycobacterium tuberculosis
ALLVRMPTSLPAVA

SEQ ID NO: 80 - protein, 27 aa, Mycobacterium tuberculosis
SRLRTHVRPDAPLVPLALRVDGLRSRW

SEQ ID NO: 81 - protein, 14 aa, Mycobacterium tuberculosis
AAVLTMLGVAGYGW

SEQ ID NO: 82 - protein, 21 aa, Mycobacterium tuberculosis
GLFMIFLDALIVNVALPDIQR

SEQ ID NO: 83 - protein, 25 aa, Mycobacterium tuberculosis
SWVVASYSLGMAVFIMSAGTLADLL

SEQ ID NO: 84 - protein, 25 aa, Mycobacterium tuberculosis
QQWNFAGIEAAASAIQGNVTSIHSL

SEQ ID NO: 85 - protein, 13 aa, Mycobacterium tuberculosis
LAWITAVVPGLMV

SEQ ID NO: 86 - protein, 12 aa, Mycobacterium tuberculosis
GEIIFISGRLNG

SEQ ID NO: 87 - protein, 14 aa, Mycobacterium tuberculosis
SALLRRLSTCPPES

(19) United States
(12) Patent Application Publication
(10) Pub. No.: US 2025/0093352 A1
(43) Pub. Date: Mar. 20, 2025

(54) USE OF AMINO ACID SEQUENCES FROM MYCOBACTERIUM TUBERCULOSIS OR CORRESPONDING NUCLEIC ACIDS FOR DIAGNOSIS AND PREVENTION OF TUBERCULAR INFECTION, DIAGNOSTIC KIT AND VACCINE THEREFROM

(71) Applicant: Cellestis Limited, Chadstone (AU)

(72) Inventors: Francesca Mariani, Rome (IT); Massimo Amicosante, Rome (IT); Vittorio Colizzi, Rome (IT); Cesare Saltini, Grottaferrata (IT)

(21) Appl. No.: 18/951,781

(22) Filed: Nov. 19, 2024

Related U.S. Application Data
(63) Continuation of application No. 17/576,308, filed on Jan. 14, 2022, now Pat. No. 12,181,476.
Application No. 17/576,308 is a division of application No. 16/999,727, filed on Aug. 21, 2020, now Pat. No. 11,275,086.
Application No. 16/999,727 is a division of application No. 16/557,536, filed on Aug. 30, 2019, now Pat. No. 10,788,491.
Application No. 16/557,536 is a division of application No. 15/793,746, filed on Oct. 25, 2017, now Pat. No. 10,408,833.
Application No. 15/793,746 is a division of application No. 14/798,241, filed on Jul. 13, 2015, now Pat. No. 9,829,490.
Application No. 14/798,241 is a division of application No. 13/811,906, filed on Jun. 5, 2013, now Pat. No. 9,110,061.

(30) Foreign Application Priority Data
Jul. 23, 2010 (IT) RM2010A000411

(51) Int. Cl. G01N 33/569; A61K 38/10; A61K 38/16; C07K 7/08; C07K 14/35; G01N 33/68

(52) U.S. Cl. CPC G01N 33/56972; A61K 38/10; A61K 38/164; C07K 7/08; C07K 14/35; G01N 33/5695; G01N 33/6866; G01N 2333/35; G01N 2333/57

(57) ABSTRACT

The present invention refers to the use of gene sequences or portions thereof characterized in that the same belong to the classes of in vitro and ex vivo induced, repressed or conserved genes in Mycobacterium tuberculosis currently infected human macrophages and to corresponding peptides or consensus peptides or proteins for the preparation of specific bio-markers for the diagnosis and prevention of active or latent disease.

### RELATED APPLICATION DATA

This application is a continuation of U.S. application Ser. No. 17/576,308, filed Jan. 14, 2022, which is a divisional of U.S. application Ser. No. 16/999,727, filed Aug. 21, 2020, now U.S. Pat. No. 11,275,086, which is a divisional of U.S. patent application Ser. No. 16/557,536, filed Aug. 30, 2019, now U.S. Pat. No. 10,788,491, which is a divisional of Ser. No. 15/793,746, filed Oct. 25, 2017, now U.S. Pat. No. 10,408,833, which is a divisional of U.S. application Ser. No. 14/798,241, filed Jul. 13, 2015, now U.S. Pat. No. 9,829,490, which is a divisional of U.S. application Ser. No. 13/811,906, filed Jun. 5, 2013, now U.S. Pat. No. 9,110,061, which is a U.S. national stage application of PCT/IT2011/000266, filed Jul. 25, 2011, which claims benefit of Italian Application No. RM2010A000411, filed Jul. 23, 2010. U.S. application Ser. No. 17/576,308 is herein incorporated by reference in its entirety.

### REFERENCE TO AN ELECTRONIC SEQUENCE LISTING

The contents of the electronic sequence listing (830109_424C1_SL.xml; Size: 77,766 bytes; and Date of Creation: Oct. 14, 2024) is herein incorporated by reference in its entirety.

The present invention concerns the use of amino acid sequences from Mycobacterium tuberculosis or corresponding nucleic acids thereof for diagnosis and prevention of tubercular infection, diagnostic kit and vaccine therefrom.

More particularly the invention refers to the use of gene sequences or portions thereof characterized in that the same belong to the classes of in vitro and ex vivo induced, repressed or conserved genes in Mycobacterium tuberculosis currently infected human macrophages and corresponding peptides or consensus peptides or proteins for the preparation of specific bio-markers for diagnosis and prevention of active or latent disease.

The laboratory diagnosis of tubercular infection and active disease onset is a very important in order to guarantee specificity, rapidity and effectiveness of the therapeutic treatment.

As to the active tubercular disease, current diagnostic protocols are based on microscopic, culture or molecular methods. With reference to microscopic examination, high mycobacteria concentrations in biological sample (from 5 to 10000/ml) are needed in order a positive result to be obtained and the sensitivity is generally lower than 60% with reduced specificity due to the inability of the test to discriminate MTB and not tubercular mycobacteria.

As to culture tests, the use thereof does not assure the medical report to be carried out within acceptable time period. In fact, MTB colonies are visible only 10-24 days after the seed thereof in solid or liquid culture media (1), as the bacterium grows slowly in vitro. Furthermore, culture tests, although considered highly sensitive, produce about 10-30% rate of negative false results and are expensive.

Molecular biology tests are highly sensitive and specific, with a sensitivity comparable to culture tests and the ability of quickly discrimination of MTB and not tubercular mycobacteria. However, the sensitivity thereof is meaningfully lower for samples containing low mycobacteria concentration, that is those negatives to microscopic examination, moreover only highly specialized laboratories can use the same being thus very expensive.

Tuberculosis is still a world-wide public health emergency lacking necessary economic resources in order an efficient diagnosis and treatment program to be planned in high incidence countries and being hardly diagnosable in affected population segments in low incidence industrialized countries.

Currently, in high incidence countries, TB active diagnosis is based on MTB microscopic (40-90% sensitivity) and culture (70-90% sensitivity) examination with 2-6 week waiting times for culture tests. In low incidence industrialized countries, TB active diagnosis is based on microscopic and culture tests and, for not tubercular MTB and mycobacteria discrimination or microscopically negative samples, on molecular tests (70-90% sensitivity).

As to the diagnosis of latent or not active tubercular infection, traditional diagnostic test is tuberculin skin test, an in vivo economic and quick diagnostic test that, standardized in years '50 allows the possibility the infection to be detected carefully and quickly thus allowing essential epidemiological surveys about tubercular infection incidence and prevalence to be carried out. Tuberculin test from the point of view of public health allowed infection incidence and prevalence to be monitored in order a global disease control to be obtained and, from the point of view of preventive and clinical medicine, infected contacts by active TB bearers to be identified allowing to establish therapeutic protections against tubercular infection aiming to prevent new case onsets. Therefore latent infection diagnosis is a fundamental element of the fight against the tuberculosis both in high and low incidence countries

Various cellular and molecular immunological issues demonstrated that the contact with MTB or antigens thereof in vitro elicits a strong cell-mediated response characterized by high production of interferon-γ (IFN-γ). This suggested that the identification of IFN-γ releasing T-lymphocytes or measure of cytokine itself, as response to the mycobacterium or antigen thereof, represents a way in order already occurred infection to be diagnosed equivalently to tuberculin test.

With respect to this issue recently two kits for tubercular infection diagnosis, i.e. QuantiFERON-TB Gold and T-SPOT TB, have been commercialized, said kits using proteins or peptides based on MTB genes, belonging to MTB genome RD1 differentiation region, in order to stimulate the IFN-γ production in T lymphocytes of circulating blood. The cost of kits is quite high in order to be used on wide scale in tuberculosis high incidence countries. The sensitivity of two commercial kits is similar, being sensitivity and specificity range from 70 to 90% and from 80 to 95%, respectively. (2). Moreover, sensitivity and specificity of these kits used in Tuberculosis high incidence areas as compared to traditionally used tuberculin test are still debated. In fact, there are cases wherein the skin test proves to be even more sensitive than Quantiferon (3) and cases wherein the two tests are exactly comparable (4).

Major limits of still more widely used tuberculin skin test, carried out according to multi puncture method or Mantoux intradermal injection, are operating complexity and insufficient specificity. In fact, the test involves a first and a second patient visit for tuberculin injection and test reading by specialized sanitary staff, respectively. For the staff the contamination risk by intradermal injection syringes in case of TB-HIV co-infection occurs. As to the tuberculin test specificities, it is known that purified tuberculin protein derivative (Purified protein derivative, PPD) displays cross-reactivity with M. bovis Bacillus Calmette-Guerin (BCG) used for anti-tubercular vaccination and various environmental not tubercular mycobacteria, displaying high sequence homology with Kock bacillus genome (5, 6). Therefore, BCG vaccinated or also subjects recently in contact with MTB virulent species, i.e. as in research laboratories named, collection strain H37Rv (ATCC 27294), will be tuberculin test positive, even if with lower reaction intensity than MTB infected subjects. For this reason, positivity intensity limits discriminating MTB reactivity from that resulting from anti M. bovis BCG or non-tubercular mycobacteria immunity, have been established.

Such intensity limits, however, are not sufficiently specific in order the tubercular infection in a vaccinated or environmental mycobacteria extensively exposed population to be diagnosed.

With respect to prevention of MTB infection the unique currently available vaccine for tubercular disease prophylaxis is M. bovis Bacillus Calmette-Guerin (BCG) (ATCC 27291), a vaccine based on an avirulent M. bovis strain, used all over the world from approximately 75 years.

In the light of above therefore it is apparent the need to provide for new diagnostic kits and vaccines based on specific peptide use suitable to overcome the disadvantages of up to now known art.

There are several studies correlating peripheral blood lymphocyte response to MTB proteins and peptides in the presence of latent infection, or recent contact with TB patients, and tubercular active disease using ELISPOT analysis for the detection of blood mononucleated cells suitable to produce IFN-γ as a result of stimulation (7-15). ELISPOT is a technique allowing, by stimulation of mononucleated blood cells on culture plate using sensitizing antibodies directed against cytokine (for example IFN-γ), the frequency of cytokine producing T lymphocytes as a response to stimulation with one or more antigens, which can be proteins, peptides or other target molecules, to be detected.

T lymphocytes recognize the antigen using the antigen T cell receptor (TCR) when the antigen is presented in peptide form (8-12 amino acid long), representing the epitope, bound to a molecule of histocompatibility major complex (MHC), a receptor family expressed on the plasmatic membrane of all the nucleated cells (as to class I MHC molecules) and antigen presenting cells like dendritic cells, macrophages etc. (as to class II MHC molecules). In humans MHC system is represented by various isotype variants, HLA-A, HLA-B and HLA-C for class I molecules; HLA-DP, HLA-DQ and HLA-DR for class II molecules. Each of said molecules displays a different number of allelic variants. Since T cell TCR recognized antigen repertoire is related to the ability of subject antigen presenting cell MHC receptors to bind peptides derived from the digestion of antigenic proteins, the indispensable condition in order the peptide to be recognized as antigen and therefore suitable to activate antigenic protein specific T lymphocytes is the susceptibility thereof to be bound by MHC receptors.

Genes encoding for HLA molecules are among most polymorphic genes occurring in human genome. It is noteworthy in this context the fact that many differences among individual allelic products of these molecules are variations of bases encoding for amino acid sequence modifications of regions involved in antigenic peptide binding. These sequence variations determine the binding properties of different allelic variants of HLA molecules and therefore the antigenic peptide repertoire with which said allelic variant will be able to form a tri-molecular complex together with T lymphocytes TCR and activate said lymphocytes.

In the context of MTB protein antigen recognizing, it is to be pointed out that every single mycobacterial epitope can be bound to one or more HLA allelic variants, but not necessarily all allelic variants expressed in a population. Moreover, since each not homozigote subject expresses at least two allelic variants of HLA-A, HLA-B, HLA-C, HLA-DP, HLA-DQ and two to four HLA-DR variants, in various subjects comprising the population under investigation different epitopes can be recognized in the context of the same or different allelic variants of different isotypes.

It is therefore apparent that the peptide set suitable to bind various allelic variants of HLA isotypes expressed by every single subject in a population can be different. From above it results the need to use an antigenic peptide set suitable to represent in as exhaustive as possible way MTB peptide epitope repertoire recognized by T lymphocytes from subjects of the population under investigation. In this context it is to be pointed out that, although the whole MTB genome has been sequenced and is available to be studied, the antigens up to now used in immunological tests for tubercular infection diagnosis result from research about mycobacterium biochemical characterization during the in vitro growth step thereof in culture media and not from ex vivo experiments.

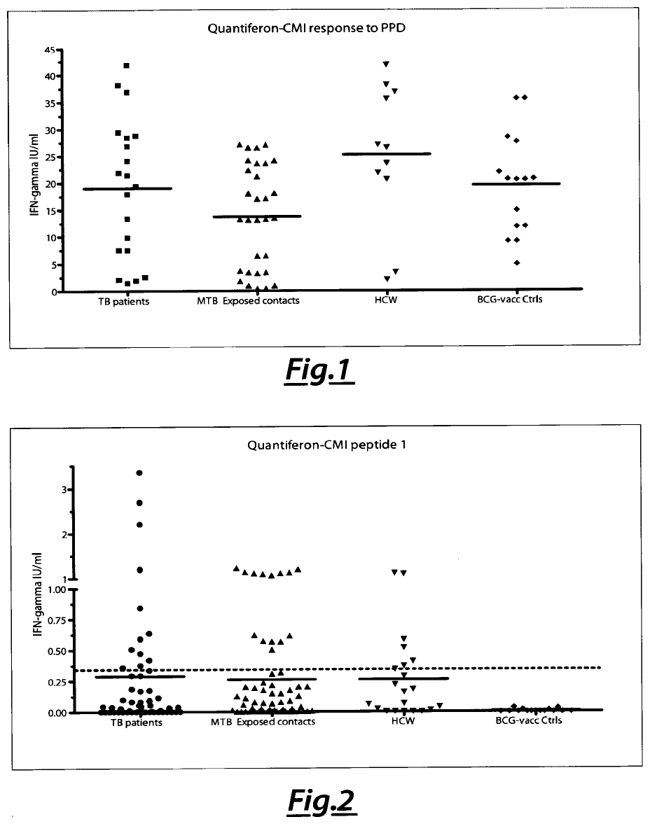

Patent application US2006/0115847 discloses an immunological diagnostic method for M. tuberculosis infection based on the combination of epitopes from proteins encoded by M. tuberculosis genome regions not occurring in BCG vaccine or most common not tubercular mycobacteria. The experimental part of said patent application reports results about various tested proteins, however the distribution of patient responses to single peptides is quite not homogeneous, in fact, as FIG. 1 shows, several TB patients are peptide insensitive. As to the patient response frequency to selected peptides, in table 6 it is shown that best individually tested peptide CFP10 induces a response in 10/15 patients, i.e. a 66.66% response frequency. Further, by means of combination of some peptides, as shown in table 7 of the US patent, sensitivity results of 92% for latent TB, i.e. PPD+ patients, 88% for active TB and 90% for TB patients under antibiotic treatment, have been obtained.

In prior studies, the authors of the present invention have identified a group of genes preferentially transcribed by human macrophages infecting MTB and characterized in that said genes belong to deletion regions of M. bovis-BCG vaccine species (WO 2005/021790).

The authors of the present invention now have analyzed proteins expressed by MTB in human macrophages, both in in vitro primary cultures, and or ex vivo in bronchoalveolar lavage (BAL) samples from active pulmonary TB patients. Using a software developed by the applicants allowing the ability of class II histocompatibility molecules to bind peptides of whole MTB genome to be analyzed, some proteins, proved to be remarkably effective from the immunological point of view, have been selected. In summary, the study compared M. tuberculosis gene expression in three different growth environments: synthetic medium culture (Sauton's), monocyte-derived-human macrophage(MDM) infected in vitro with M. tuberculosis, alveolar macrophage (AM) from bronchoalveolar lavage (BAL) samples of pulmonary TB affected patients before the antibiotic therapy.

From thus obtained 9 gene groups, first 100 proteins, according to a combination criteria (modulation of expression, immunogenicity, tubercular Complex specificity, etc), have been selected. From these 100 proteins again a 30 protein group has been selected, for which a positive response in immunological tests on TB patients whole blood had been obtained (see table 1).

After an ulterior selection from 4 groups of subjects: Pulmonary TB before antibiotic therapy (n=13), recently exposed healthy contacts (TB patient relatives) PPD+ (n=8); long term TB patient exposed healthy contacts (professional exposure of hospital workers) PPD+ (n=5); BCG vaccinated negative controls, PPD− (n=4), firstly 43 peptides have been designed, synthesized and tested.

Then the 6 most sensible and specific peptides have been selected (see table 2), and the study has been repeated using an extended subject sample (see tables 3-5 and FIGS. 1-7).

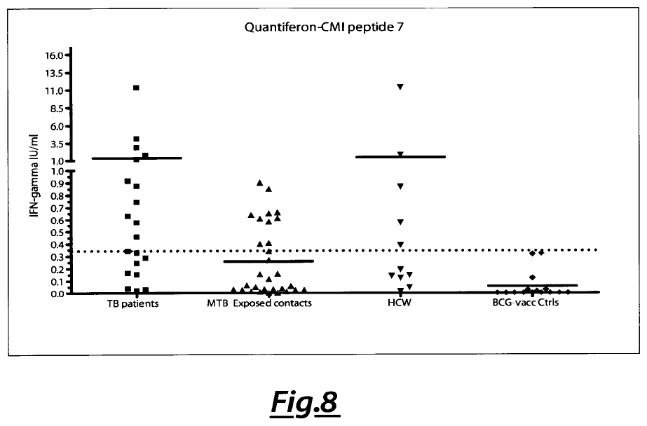

The results obtained using said six peptides and a peptide belonging to ESAT6, i.e an highly immunogenic protein occurring in both above mentioned commercial kits (FIG. 8), have been compared.

In summary, all the selected peptides displayed T-cell reactivity. Particularly, peptide #3 (SEQ ID NO:71 of the present invention) displays an elevated sensitivity comparable, if not more elevated, to multiepitope ESAT-6 protein derived control peptide. It is noteworthy that, as in panel estimated (i.e. by estimating ex-post total data of each individual peptide), 6 multiepitope peptides are recognized by about 75% of the subjects with active TB (a result comparable, in this series, to Quantiferon-TB gold in-Tube, as shown in Table 4). Data are perfectly similar to the result obtained in a patient subgroup tested using said 6 peptides concurrently in the same well (as shown in Table 5).

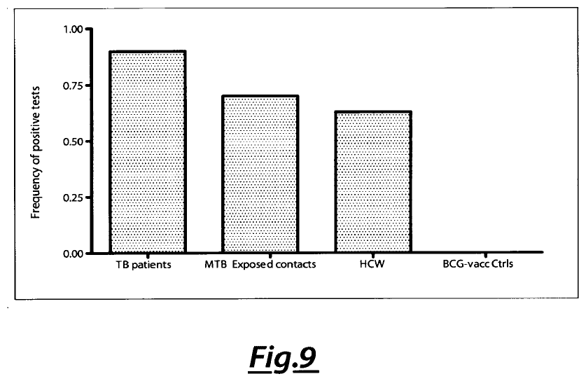

Peptide optimal diagnostic sensitivity is associated together with an optimal specificity. In fact, the reported peptide response is limited to active TB subjects, recent exposure contacts and exposed sanitary staff (as shown in Table 4 and FIG. 9). It is noteworthy that, although the data must be supported by larger subject number, no peptide response from M. bovis BCG antitubercular vaccinated control subjects, not even from Quantiferon-TB gold in-Tube positive 3 subjects, individually has been detected.

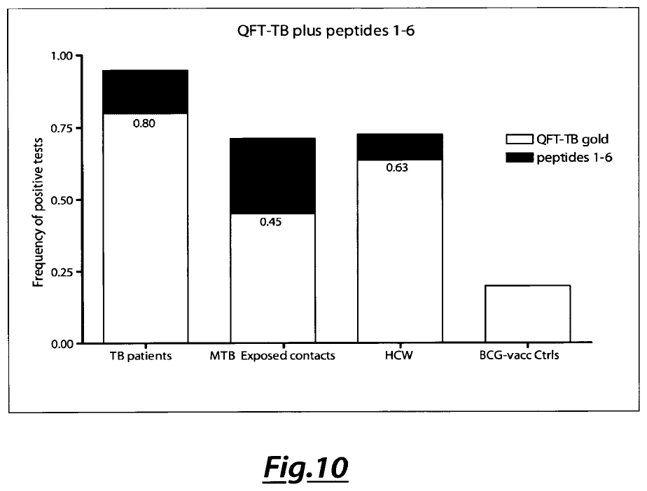

Further, the peptides allow the commercial test sensitivity, i.e. current gold reference standard for tubercular infection diagnosis (FIG. 10), to be enhanced. Both when panel estimated and directly tested within same well, 6 selected peptides allow Quantiferon-TB gold in-Tube to be enhanced from 75% to 89% (+14%) and from 71% to 83% (+13%), respectively, in subjects with active TB (see Table 4 and 5).

It is therefore a specific object of the present invention the use of at least 6 peptides, derived from Mycobacterium tuberculosis and comprising at least one T-cell epitope, in association with ESAT6 and CFP10 and, optionally with TB7.7, as biomarkers in an in vitro test for the detection of Mycobacterium tuberculosis infection in a subject, said peptides being chosen from the group consisting of: TAWITAVVPGLMV (SEQ ID NO:24), ELMARAAVLGSAH (SEQ ID NO:21), RPVRRVLLFVVPSSGPAP (SEQ ID NO:70), GSVRQLPSVLKPPLITLRTLTLSG (SEQ ID NO:71), GEIIFISGRLNGaa (SEQ ID NO:13), AVIVRSELLTQYL (SEQ ID NO:22), LAWITAVVPGLMV (SEQ ID NO:85), GEIIFISGRLNG (SEQ ID NO:86) or SALLRRLSTCPPES (SEQ ID NO:87). According to an embodiment of the present invention the peptides are the following six peptides: ELMARAAVLGSAH (SEQ ID NO:21), RPVRRVLLFVVPSSGPAP (SEQ ID NO:70), GSVRQLPSVLKPPLITLRTLTLSG (SEQ ID NO:71), AVIVRSELLTQYL (SEQ ID NO:22), TAWITAVVPGLMV (SEQ ID NO:24) and GEIIFISGRLNGaa (SEQ ID NO:13). According to a further embodiment of the present invention, the peptides are the following six peptides: ELMARAAVLGSAH (SEQ ID NO:21), RPVRRVLLFVVPSSGPAP (SEQ ID NO:70), GSVRQLPSVLKPPLITLRTLTLSG (SEQ ID NO:71), AVIVRSELLTQYL (SEQ ID NO:22), LAWITAVVPGLMV (SEQ ID NO:85) and GEIIFISGRLNG (SEQ ID NO:86). According to another embodiment of the present invention the peptides are all the following nine peptides: TAWITAVVPGLMV (SEQ ID NO:24), ELMARAAVLGSAH (SEQ ID NO:21), RPVRRVLLFVVPSSGPAP (SEQ ID NO:70), GSVRQLPSVLKPPLITLRTLTLSG (SEQ ID NO:71), GEIIFISGRLNGaa (SEQ ID NO:13), AVIVRSELLTQYL (SEQ ID NO:22), LAWITAVVPGLMV (SEQ ID NO:85), GEIIFISGRLNG (SEQ ID NO:86) and SALLRRLSTCPPES (SEQ ID NO:87).

The present invention concerns also the use of at least one peptide, derived from Mycobacterium tuberculosis and comprising at least one T-cell epitope, in association with ESAT6 and CFP10 and, optionally, with TB7.7, as biomarker in an in vitro test for the detection of Mycobacterium tuberculosis infection in a subject, said peptide being chosen from the group consisting of: TAWITAVVPGLMV (SEQ ID NO:24), ELMARAAVLGSAH (SEQ ID NO:21), RPVRRVLLFVVPSSGPAP (SEQ ID NO:70), GSVRQLPSVLKPPLITLRTLTLSG (SEQ ID NO:71), GEIIFISGRLNGaa (SEQ ID NO:13), AVIVRSELLTQYL (SEQ ID NO:22), LAWITAVVPGLMV (SEQ ID NO:85), GEIIFISGRLNG (SEQ ID NO:86) or SALLRRLSTCPPES (SEQ ID NO: 87). Particularly said at least one peptide can be LAWITAVVPGLMV (SEQ ID NO:85) or TAWITAVVPGLMV (SEQ ID NO:24).

It is further object of the present invention a method for in vitro diagnosing infection by a Mycobacterium tuberculosis in a subject,
said method comprising incubating a blood sample comprising lymphocytes from said subject in the presence of at least six peptides, derived from Mycobacterium tuberculosis and comprising at least one T-cell epitope, in association with ESAT6 and CFP10 and, optionally with TB7.7,
for a time and under conditions sufficient to stimulate the lymphocytes to produce an effector molecule, wherein the presence or level of the effector molecule is indicative of the lymphocytes derived from a subject infected with or prior exposed to the Mycobacterium species,
said at least six peptides being chosen from the group consisting of TAWITAVVPGLMV (SEQ ID NO:24), ELMARAAVLGSAH (SEQ ID NO:21), RPVRRVLLFVVPSSGPAP (SEQ ID NO:70), GSVRQLPSVLKPPLITLRTLTLSG (SEQ ID NO:71), GEIIFISGRLNGaa (SEQ ID NO:13), AVIVRSELLTQYL (SEQ ID NO:22), LAWITAVVPGLMV (SEQ ID NO:85), GEIIFISGRLNG (SEQ ID NO:86) or SALLRRLSTCPPES (SEQ ID NO:87).

According to an embodiment of the method of the present invention, the peptides are the following six peptides: ELMARAAVLGSAH (SEQ ID NO:21), RPVRRVLLFVVPSSGPAP (SEQ ID NO:70), GSVRQLPSVLKPPLITLRTLTLSG (SEQ ID NO:71), AVIVRSELLTQYL (SEQ ID NO:22), TAWITAVVPGLMV (SEQ ID NO:24) and GEIIFISGRLNGaa (SEQ ID NO:13). According to a further embodiment of the method of the present invention, the peptides are the following six peptides: ELMARAAVLGSAH (SEQ ID NO:21), RPVRRVLLFVVPSSGPAP (SEQ ID NO:70), GSVRQLPSVLKPPLITLRTLTLSG (SEQ ID NO:71), AVIVRSELLTQYL (SEQ ID NO:22), LAWITAVVPGLMV (SEQ ID NO:85) and GEIIFISGRLNG (SEQ ID NO:86). According to anther embodiment, the peptides are all the following nine peptides: TAWITAVVPGLMV (SEQ ID NO:24), ELMARAAVLGSAH (SEQ ID NO:21), RPVRRVLLFVVPSSGPAP (SEQ ID NO:70), GSVRQLPSVLKPPLITLRTLTLSG (SEQ ID NO:71), GEIIFISGRLNGaa (SEQ ID NO:13), AVIVRSELLTQYL (SEQ ID NO:22), LAWITAVVPGLMV (SEQ ID NO:85), GEIIFISGRLNG (SEQ ID NO:86) and SALLRRLSTCPPES (SEQ ID NO:87).

It is an object of the present invention a method for in vitro diagnosing infection by a Mycobacterium tuberculosis in a subject,
said method comprising incubating a blood sample comprising lymphocytes from said subject in the presence of at least one peptide, derived from Mycobacterium tuberculosis and comprising at least one T-cell epitope, in association with ESAT6 and CFP10 and, optionally, with TB7.7,
for a time and under conditions sufficient to stimulate the lymphocytes to produce an effector molecule, wherein the presence or level of the effector molecule is indicative of the lymphocytes derived from a subject infected with or prior exposed to the Mycobacterium species,
said at least one peptide being chosen from the group consisting of TAWITAVVPGLMV (SEQ ID NO:24), ELMARAAVLGSAH (SEQ ID NO:21), RPVRRVLLFVVPSSGPAP (SEQ ID NO:70), GSVRQLPSVLKPPLITLRTLTLSG (SEQ ID NO:71), GEIIFISGRLNGaa (SEQ ID NO:13), AVIVRSELLTQYL (SEQ ID NO:22), LAWITAVVPGLMV (SEQ ID NO:85), GEIIFISGRLNG (SEQ ID NO:86) or SALLRRLSTCPPES (SEQ ID NO:87). For example, said at least one peptide can be LAWITAVVPGLMV (SEQ ID NO:85) or TAWITAVVPGLMV (SEQ ID NO:24).

It is further object of the present invention a method for the in vitro diagnosis of infection by Mycobacterium tuberculosis in a subject, said method including incubating a blood sample comprising lymphocytes from the subject with ESAT6 and CFP10, and optionally with TB7.7, and measuring release of interferon-γ by the lymphocytes, said method being characterized in that the incubation is carried out further in the presence of at least six peptides chosen from the group consisting of TAWITAVVPGLMV (SEQ ID NO:24), ELMARAAVLGSAH (SEQ ID NO:21), RPVRRVLLFVVPSSGPAP (SEQ ID NO:70), GSVRQLPSVLKPPLITLRTLTLSG (SEQ ID NO:71), GEIIFISGRLNGaa (SEQ ID NO:13), AVIVRSELLTQYL (SEQ ID NO:22), LAWITAVVPGLMV (SEQ ID NO:85), GEIIFISGRLNG (SEQ ID NO:86) or SALLRRLSTCPPES (SEQ ID NO:87), wherein the level of sensitivity and/or selectivity for the detection of Mycobacterium tuberculosis is higher compared to the sensitivity and/or selected using ESAT6 and CFP10, optionally with TB7.7. According to an embodiment of the present invention, the peptides are the following six peptides: ELMARAAVLGSAH (SEQ ID NO:21), RPVRRVLLFVVPSSGPAP (SEQ ID NO:70), GSVRQLPSVLKPPLITLRTLTLSG (SEQ ID NO:71), AVIVRSELLTQYL (SEQ ID NO:22), TAWITAVVPGLMV (SEQ ID NO:24) and GEIIFISGRLNGaa (SEQ ID NO:13). According to a further embodiment the peptides are the following six peptides: ELMARAAVLGSAH (SEQ ID NO:21), RPVRRVLLFVVPSSGPAP (SEQ ID NO:70), GSVRQLPSVLKPPLITLRTLTLSG (SEQ ID NO:71), AVIVRSELLTQYL (SEQ ID NO:22), LAWITAVVPGLMV (SEQ ID NO:85) and GEIIFISGRLNG (SEQ ID NO:86). According to another embodiment the peptides are all the following nine peptides: TAWITAVVPGLMV (SEQ ID NO:24), ELMARAAVLGSAH (SEQ ID NO:21), RPVRRVLLFVVPSSGPAP (SEQ ID NO:70), GSVRQLPSVLKPPLITLRTLTLSG (SEQ ID NO:71), GEIIFISGRLNGaa (SEQ ID NO:13), AVIVRSELLTQYL (SEQ ID NO:22), LAWITAVVPGLMV (SEQ ID NO:85), GEIIFISGRLNG (SEQ ID NO:86) and SALLRRLSTCPPES (SEQ ID NO:87).

The present invention concerns also a method for the in vitro diagnosis of infection by Mycobacterium tuberculosis in a subject, said method including incubating a blood sample comprising lymphocytes from the subject with ESAT6 and CFP10, and optionally with TB7.7, and measuring release of interferon-γ by the lymphocytes, said method being characterized in that the incubation is carried out further in the presence of at least one peptide chosen from the group consisting of TAWITAVVPGLMV (SEQ ID NO:24), ELMARAAVLGSAH (SEQ ID NO:21), RPVRRVLLFVVPSSGPAP (SEQ ID NO:70), GSVRQLPSVLKPPLITLRTLTLSG (SEQ ID NO:71), GEIIFISGRLNGaa (SEQ ID NO:13), AVIVRSELLTQYL (SEQ ID NO:22), LAWITAVVPGLMV (SEQ ID NO:85), GEIIFISGRLNG (SEQ ID NO:86) or SALLRRLSTCPPES (SEQ ID NO:87), wherein the level of sensitivity and/or selectivity for the detection of Mycobacterium tuberculosis is higher compared to the sensitivity and/or selected using ESAT6 and CFP10, optionally with TB7.7. Said at least one peptide can be LAWITAVVPGLMV (SEQ ID NO:85) or TAWITAVVPGLMV (SEQ ID NO:24).

It is further object of the present invention the use of at least one biomarker selected from the list consisting of:
(i) a protein selected from Rv0023, Rv0182c, Rv0290, Rv0601c, Rv0647c, Rv0724A, Rv0890c, Rv1251c, Rv1398c, Rv1478, Rv1497, Rv1575, Rv1578c, Rv1899c, Rv2137c, Rv2333c, Rv2548, Rv2557, Rv2816c, Rv2990, Rv3094c, Rv3107c, Rv3188, Rv3239c, Rv3296, Rv3425, Rv3446c, Rv3479, Rv3482c, Rv3780, derived from a Mycobacterium species or related organism and comprising at least one T-cell epitope;
(ii) a homolog of the protein as defined in (i) having an amino acid sequence with at least 80% similarity in comparison to one of said protein after optimal alignment; and
(iii) a peptide fragment of the protein as defined in (i) or (ii) having a T-cell epitope or a chemical analog thereof;
in an in vitro test for the detection of Mycobacterium infection in a subject, i.e., a human or non-human animal subject.

The Mycobacterium species can be selected from M. tuberculosis, M. bovis, M. bovis BCG, M. africanum, M. canetti, M. caprae, M. microti, M. pinnipedii, M. avium, M. avium paratuberculosis, M. avium silvaticum, M. avium “hominissuis”, M. colombiense, M. asiaticum, M. gordonae, M. gastri, M. kansasii, M. hiberniae, M. nonchromogenicum, M. terrae, M. triviale, M. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. simiae, M. branderi, M. cookii, M. celatum, M. bohemicum, M. haemophilus. malmoense, M. szulgai, M. leprae, M. lepraemurium, M. lepromatosis, M. africanum, M. botniense, M. chimaera, M. conspicuum, M. doricum, M. farcinogenes, M. heckeshornense, M. intracellulare, M. lacus, M. marinum, M. monacense, M. montefiorense, M. murale, M. nebraskense, M. saskatchewanense, M. scrofulaceum, M. shimoidei, M. tusciae, M. xenopi, M. intermedium, M. abscessus, M. chelonae, M. bolletii, M. fortuitum, M. fortuitum subsp. acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diernhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodii, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, M. seoulense and M. tokaiense. Preferably, the Mycobacterium species is Mycobacterium tuberculosis.

According to the above use, the peptide fragment as defined in (iii) can comprise or consist of an amino acid sequence selected from TAWITAVVPGLMV (SEQ ID NO:24), AVIVRSELLTQYL (SEQ ID NO:22), GSVRQLPSVLKPPLITLRTLTLSG (SEQ ID NO:71), RPVRRVLLFVVPSSGPAP (SEQ ID NO:70), GEIIFISGRLNGaa (SEQ ID NO:13), ELMARAAVLGSAH (SEQ ID NO:21), LAWITAVVPGLMV (SEQ ID NO:85), GEIIFISGRLNG (SEQ ID NO:86), SALLRRLSTCPPES (SEQ ID NO:87).

The above use can further comprise the use of one or more Mycobacterium protein or peptide fragment thereof or chemical analog derived therefrom selected from ESAT6, CFP10, TB7.7 and PPD.

It is further object of the present invention the use of at least one biomarker selected from the list consisting of:
(i) a protein selected from Rv0023, Rv0182c, Rv0290, Rv0601c, Rv0647c, Rv0724A, Rv0890c, Rv1251c, Rv1398c, Rv1478, Rv1497, Rv1575, Rv1578c, Rv1899c, Rv2137c, Rv2333c, Rv2548, Rv2557, Rv2816c, Rv2990, Rv3094c, Rv3107c, Rv3188, Rv3239c, Rv3296, Rv3425, Rv3446c, Rv3479, Rv3482c, Rv3780, derived from a Mycobacterium species or related organism and comprising at least one T-cell epitope;
(ii) a homolog of the protein as defined in (i) having an amino acid sequence with at least 80% similarity to one of said protein after optimal alignment; and
(iii) a peptide fragment of the protein as defined in (i) or (ii) having a T-cell epitope or a chemical analog thereof; and
(iv) a Mycobacterium derived protein or fragment thereof or chemical analog thereof selected from the list consisting of:
(a) ESAT;
(b) CFP10;
(c) TB7.7; and
(d) PPD;
in an in vitro test for the detection of Mycobacterium infection in a subject i.e. an human or a non-human animal subject.

The Mycobacterium species can be selected from M. tuberculosis, M. bovis, M. bovis BCG, M. africanum, M. canetti, M. caprae, M. microti, M. pinnipedii, M. avium, M. avium paratuberculosis, M. avium silvaticum, M. avium “hominissuis”, M. colombiense, M. asiaticum, M. gordonae, M. gastri, M. kansasii, M. hiberniae, M. nonchromogenicum, M. terrae, M. triviale, M. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. simiae, M. branderi, M. cookii, M. celatum, M. bohemicum, M. haemophilus, M. malmoense, M. szulgai, M. leprae, M. lepraemurium, M. lepromatosis, M. africanum, M. botniense, M. chimaera, M. conspicuum, M. doricum, M. farcinogenes, M. heckeshornense, M. intracellulare, M. lacus, M. marinum, M. monacense, M. montefiorense, M. murale, M. nebraskense, M. saskatchewanense, M. scrofulaceum, M. shimoidei, M. tusciae, M. xenopi, M. intermedium, M. abscessus, M. chelonae, M. bolletii, M. fortuitum, M. fortuitum subsp. acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diernhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodii, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, M. seoulense and M. tokaiense. Preferably, the Mycobacterium species is Mycobacterium tuberculosis.

According to the above use, the peptide fragment as defined in (iii) can comprise or consist of an amino acid sequence selected from TAWITAVVPGLMV (SEQ ID NO:24), AVIVRSELLTQYL (SEQ ID NO:22), GSVRQLPSVLKPPLITLRTLTLSG (SEQ ID NO:71), RPVRRVLLFVVPSSGPAP (SEQ ID NO:70), GEIIFISGRLNGaa (SEQ ID NO:13), ELMARAAVLGSAH (SEQ ID NO:21), LAWITAVVPGLMV (SEQ ID NO:85), GEIIFISGRLNG (SEQ ID NO:86), SALLRRLSTCPPES (SEQ ID NO:87).

It is a further object of the present invention the use of at least one nucleic acid molecule encoding for a biomarker selected from the list consisting of:
(i) a protein selected from Rv0023, Rv0182c, Rv0290, Rv0601c, Rv0647c, Rv0724A, Rv0890c, Rv1251c, Rv1398c, Rv1478, Rv1497, Rv1575, Rv1578c, Rv1899c, Rv2137c, Rv2333c, Rv2548, Rv2557, Rv2816c, Rv2990, Rv3094c, Rv3107c, Rv3188, Rv3239c, Rv3296, Rv3425, Rv3446c, Rv3479, Rv3482c, Rv3780, derived from a Mycobacterium species or related organism and comprising at least one T-cell epitope;
(ii) a homolog of the protein as defined in (i) having an amino acid sequence with at least 80% similarity to one of said protein after optimal alignment; and
(iii) a peptide fragment of the protein as defined in (i) or (ii) having a T-cell epitope or chemical analog thereof;
   in an in vitro test for the detection of Mycobacterium infection in a subject i.e. an human or a non-human animal subject.

The Mycobacterium species is selected from M. tuberculosis, M. bovis, M. bovis BCG, M. africanum, M. canetti, M. caprae, M. microti, M. pinnipedii, M. avium, M. avium paratuberculosis, M. avium silvaticum, M. avium “hominissuis”, M. colombiense, M. asiaticum, M. gordonae, M. gastri, M. kansasii, M. hiberniae, M. nonchromogenicum, M. terrae, M. triviale, M. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. simiae, M. branderi, M. cookii, M. celatum, M. bohemicum, M. haemophilus. malmoense, M. szulgai, M. leprae, M. lepraemurium, M. lepromatosis, M. africanum, M. botniense, M. chimaera, M. conspicuum, M. doricum, M. farcinogenes, M. heckeshornense, M. intracellulare, M. lacus, M. marinum, M. monacense, M. montefiorense, M. murale, M. nebraskense, M. saskatchewanense, M. scrofulaceum, M. shimoidei, M. tusciae, M. xenopi, M. intermedium, M. abscessus, M. chelonae, M. bolletii, M. fortuitum, M. fortuitum subsp. acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diernhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodii, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, M. seoulense and M. tokaiense. Preferably, the Mycobacterium species is Mycobacterium tuberculosis.

According to the above use, the peptide fragment in (iii) can comprise or consist of an amino acid sequence selected from TAWITAVVPGLMV (SEQ ID NO:24), AVIVRSELLTQYL (SEQ ID NO:22), GSVRQLPSVLKPPLITLRTLTLSG (SEQ ID NO:71), RPVRRVLLFVVPSSGPAP (SEQ ID NO:70), GEIIFISGRLNGaa (SEQ ID NO:13), ELMARAAVLGSAH (SEQ ID NO:21), LAWITAVVPGLMV (SEQ ID NO:85), GEIIFISGRLNG (SEQ ID NO:86), SALLRRLSTCPPES (SEQ ID NO:87). Said use can further comprise use of a nucleic acid molecule encoding for a Mycobacterium protein or peptide fragment derived therefrom selected from ESAT6, CFP10, TB7.7 and PPD or an homolog thereof.

It is further object of the present invention, an isolated protein selected from the list consisting of Rv0023, Rv0182c, Rv0290, Rv0601c, Rv0647c, Rv0724A, Rv0890c, Rv1251c, Rv1398c, Rv1478, Rv1497, Rv1575, Rv1578c, Rv1899c, Rv2137c, Rv2333c, Rv2548, Rv2557, Rv2816c, Rv2990, Rv3094c, Rv3107c, Rv3188, Rv3239c, Rv3296, Rv3425, Rv3446c, Rv3479, Rv3482c, Rv3780,derived from a Mycobacterium species and comprising a T-cell epitope. The isolated peptide of the protein according to the invention can comprise a T-cell epitope or a chemical analog thereof. The isolated peptide according can comprise or consist of an amino acid sequence selected from TAWITAVVPGLMV (SEQ ID NO:24), AVIVRSELLTQYL (SEQ ID NO:22), GSVRQLPSVLKPPLITLRTLTLSG (SEQ ID NO:71), RPVRRVLLFVVPSSGPAP (SEQ ID NO:70), GEIIFISGRLNGaa (SEQ ID NO:13), ELMARAAVLGSAH (SEQ ID NO:21), LAWITAVVPGLMV (SEQ ID NO:85), GEIIFISGRLNG (SEQ ID NO:86), SALLRRLSTCPPES (SEQ ID NO:87).

It is further object of the present invention an isolated nucleic acid molecule encoding for the above-mentioned protein or peptide according to the present invention.

The present invention concerns a vector comprising the above mentioned nucleic acid molecule, and isolated cell comprising said vector.

It is a further object of the present invention a kit comprising a container, said container comprising the above mentioned at least one protein or at least one a peptide or at least one a nucleic acid molecule.

The present invention concerns a method for in vitro diagnosing infection by a Mycobacterium species in a subject, said method comprising incubating a blood sample comprising lymphocytes from said subject in the presence of at least one biomarker selected from the list consisting of:
(i) a protein selected from Rv0023, Rv0182c, Rv0290, Rv0601c, Rv0647c, Rv0724A, Rv0890c, Rv1251c, Rv1398c, Rv1478, Rv1497, Rv1575, Rv1578c, Rv1899c, Rv2137c, Rv2333c, Rv2548, Rv2557, Rv2816c, Rv2990, Rv3094c, Rv3107c, Rv3188, Rv3239c, Rv3296, Rv3425, Rv3446c, Rv3479, Rv3482c, Rv3780, derived from a Mycobacterium species or related organism and comprising at least one T-cell epitope;
(ii) a homolog of the protein as defined in (i) having an amino acid sequence with at least 80% similarity to one of said protein after optimal alignment; and
(iii) a peptide fragment of the protein as defined in (i) or (ii) having a T-cell epitope or a chemical analog thereof;
for a time and under conditions sufficient to stimulate the lymphocytes to produce an effector molecule, wherein the presence or level of the effector molecule is indicative of the lymphocytes derived from a subject infected with or prior exposed to the Mycobacterium species. The subject can be an human or a non-human animal. The incubation between blood and biomarker can occur in a test-tube, optionally in the presence of heparin, in the presence of an added carbohydrate. According to an embodiment of the present invention, the incubation can occur further in the presence of a Mycobacterium protein or peptide fragment thereof or chemical analog thereof derived therefrom selected from ESAT6, CFP10, TB7.7 and PPD or mixture thereof. The Mycobacterium species is selected from M. tuberculosis, M. bovis, M. bovis BCG, M. africanum, M. canetti, M. caprae, M. microti, M. pinnipedii, M. avium, M. avium paratuberculosis, M. avium silvaticum, M. avium “hominissuis”, M. colombiense, M. asiaticum, M. gordonae, M. gastri, M. kansasii, M. hiberniae, M. nonchromogenicum, M. terrae, M. triviale, M. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. simiae, M. branderi, M. cookii, M. celatum, M. bohemicum, M. haemophilu malmoense, M. szulgai, M. leprae, M. lepraemurium, M. lepromatosis, M. africanum, M. botniense, M. chimaera, M. conspicuum, M. doricum, M. farcinogenes, M. heckeshornense, M. intracellulare, M. lacus, M. marinum, M. monacense, M. montefiorense, M. murale, M. nebraskense, M. saskatchewanense, M. scrofulaceum, M. shimoidei, M. tusciae, M. xenopi, M. intermedium, M. abscessus, M. chelonae, M. bolletii, M. fortuitum, M. fortuitum subsp. acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diernhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodii, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, M. seoulense and M. tokaiense. Preferably, the Mycobacterium species is Mycobacterium tuberculosis.

According to the above-mentioned method of the invention, the peptide as defined in (iii) comprises or consists of an amino acid sequence selected from TAWITAVVPGLMV (SEQ ID NO:24), AVIVRSELLTQYL (SEQ ID NO:22), GSVRQLPSVLKPPLITLRTLTLSG (SEQ ID NO:71), RPVRRVLLFVVPSSGPAP (SEQ ID NO:70), GEIIFISGRLNGaa (SEQ ID NO:13), ELMARAAVLGSAH (SEQ ID NO:21), LAWITAVVPGLMV (SEQ ID NO:85), GEIIFISGRLNG (SEQ ID NO:86), SALLRRLSTCPPES (SEQ ID NO:87). The effector molecule can be selected from interferon-γ, a cytokine, an interleukin and TNF-α, preferably interferon-γ.

It is a further object of the present invention an isolated antibody specific for the protein or peptide defined above according.

The invention concerns also a method for the in vitro diagnosis of infection by Mycobacterium tuberculosis in a subject, said method including incubating a blood sample comprising lymphocytes from the subject with one or more of ESAT6, CFP10, TB7.7 and/or PPD and measuring release of interferon-γ by the lymphocytes, said method being characterized in that the incubation is carried out further in the presence of at least one biomarker selected from:
(i) a protein selected from Rv0023, Rv0182c, Rv0290, Rv0601c, Rv0647c, Rv0724A, Rv0890c, Rv1251c, Rv1398c, Rv1478, Rv1497, Rv1575, Rv1578c, Rv1899c, Rv2137c, Rv2333c, Rv2548, Rv2557, Rv2816c, Rv2990, Rv3094c, Rv3107c, Rv3188, Rv3239c, Rv3296, Rv3425, Rv3446c, Rv3479, Rv3482c, Rv3780, derived from a Mycobacterium species or related organism and comprising at least one T-cell epitope;
(ii) a homolog of the protein as defined in (i) having an amino acid sequence with at least 80% similarity to one of said protein after optimal alignment; and
(iii) a peptide fragment of the protein as defined in (i) or (ii) having a T-cell epitope or chemical analog thereof;
wherein the level of sensitivity and/or selectivity for the detection of Mycobacterium tuberculosis is higher compared to the sensitivity and/or selected using one or more of ESAT6, CFP10, TB7.7 and/or PPD alone.

In addition, the present invention concerns vaccine for the treatment or prophylaxis of infection by a Mycobacterium species, said vaccine comprising or consisting of at least one agent selected from the list consisting of:
(i) a protein selected from Rv0023, Rv0182c, Rv0290, Rv0601c, Rv0647c, Rv0724A, Rv0890c, Rv1251c, Rv1398c, Rv1478, Rv1497, Rv1575, Rv1578c, Rv1899c, Rv2137c, Rv2333c, Rv2548, Rv2557, Rv2816c, Rv2990, Rv3094c, Rv3107c, Rv3188, Rv3239c, Rv3296, Rv3425, Rv3446c, Rv3479, Rv3482c, Rv3780, derived from a Mycobacterium species or related organism and comprising at least one T-cell epitope;
(ii) a homolog of the protein as defined in (i) having an amino acid sequence with at least 80% similarity to one of said protein after optimal alignment; and
(iii) a peptide fragment of the protein as defined in (i) or (ii) having a T-cell epitope or chemical analog thereof;
and one or more pharmaceutically acceptable adjuvants, carriers, excipients and/or diluents.

According to the vaccine of the present invention, the Mycobacterium species is selected from M. tuberculosis, M. bovis, M. bovis BCG, M. africanum, M. canetti, M. caprae, M. microti, M. pinnipedii, M. avium, M. avium paratuberculosis, M. avium silvaticum, M. avium “hominissuis”, M. colombiense, M. asiaticum, M. gordonae, M. gastri, M. kansasii, M. hiberniae, M. nonchromogenicum, M. terrae, M. triviale, M. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. simiae, M. branderi, M. cookii, M. celatum, M. bohemicum, M. haemophilus. malmoense, M. szulgai, M. leprae, M. lepraemurium, M. lepromatosis, M. africanum, M. botniense, M. chimaera, M. conspicuum, M. doricum, M. farcinogenes, M. heckeshornense, M. intracellulare, M. lacus, M. marinum, M. monacense, M. montefiorense, M. murale, M. nebraskense, M. saskatchewanense, M. scrofulaceum, M. shimoidei, M. tusciae, M. xenopi, M. intermedium, M. abscessus, M. chelonae, M. bolletii, M. fortuitum, M. fortuitum subsp. acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diernhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodii, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, M. seoulense and M. tokaiense. Preferably, the Mycobacterium species is Mycobacterium tuberculosis.

Vaccine of the present invention can be use in human or non-human animal subject. The peptide as defined in (iii) can comprise or consist of an amino acid sequence selected from TAWITAVVPGLMV (SEQ ID NO:24), AVIVRSELLTQYL (SEQ ID NO:22), GSVRQLPSVLKPPLITLRTLTLSG (SEQ ID NO:71), RPVRRVLLFVVPSSGPAP (SEQ ID NO:70), GEIIFISGRLNGaa (SEQ ID NO:13), ELMARAAVLGSAH (SEQ ID NO:21), LAWITAVVPGLMV (SEQ ID NO:85), GEIIFISGRLNG (SEQ ID NO:86), SALLRRLSTCPPES (SEQ ID NO:87). Therefore, the present invention concerns vaccine as define above for use in the prevention of infection by Mycobacterium species.

It is further object of the present invention, at least one agent selected from the list consisting of:
(i) a protein selected from Rv0023, Rv0182c, Rv0290, Rv0601c, Rv0647c, Rv0724A, Rv0890c, Rv1251c, Rv1398c, Rv1478, Rv1497, Rv1575, Rv1578c, Rv1899c, Rv2137c, Rv2333c, Rv2548, Rv2557, Rv2816c, Rv2990, Rv3094c, Rv3107c, Rv3188, Rv3239c, Rv3296, Rv3425, Rv3446c, Rv3479, Rv3482c, Rv3780, derived from a Mycobacterium species or related organism and comprising at least one T-cell epitope;
(ii) a homolog of the protein as defined in (i) having an amino acid sequence with at least 80% similarity to one of said protein after optimal alignment; and
(iii) a peptide fragment of the protein as defined in (i) or (ii) having a T-cell epitope or a chemical analog thereof;
for use in the treatment or prevention of infection by Mycobacterium species.

The present invention further concerns a method for in vitro assessing the capacity for a subject to mount a cell-mediated immune response, said method comprising contacting a sample comprising T-lymphocytes sensitized to Mycobacterium species or antigens or proteins comprising T-lymphocyte epitopes derived thereform with at least one agent selected from:
(i) a protein selected from Rv0023, Rv0182c, Rv0290, Rv0601c, Rv0647c, Rv0724A, Rv0890c, Rv1251c, Rv1398c, Rv1478, Rv1497, Rv1575, Rv1578c, Rv1899c, Rv2137c, Rv2333c, Rv2548, Rv2557, Rv2816c, Rv2990, Rv3094c, Rv3107c, Rv3188, Rv3239c, Rv3296, Rv3425, Rv3446c, Rv3479, Rv3482c, Rv3780, derived from a Mycobacterium species or related organism and comprising at least one T-cell epitope;
(ii) a homolog of the protein as defined in (i) having an amino acid sequence with at least 80% similarity to one of said protein after optimal alignment; and
(iii) a peptide fragment of the protein as defined in (i) or (ii) having a T-cell epitope or a chemical analog thereof;
for a time and under conditions sufficient to stimulate the lymphocytes to produce an effector molecule, wherein the presence or level of the effector molecule is indicative of the subject's capacity to mount a cell-mediated immune response.

The present invention now will be described by an illustrative, but not limitative way, according to preferred embodiments thereof, with particular reference to enclosed drawings wherein:

FIG. 1. Analysis of IFN-gamma production as PPD response comparing four subject populations: a. first test pulmonary TB patients; b. healthy contacts, TB exposed, PPD positive; c. healthy controls, professionally TB exposed, Quantiferon positive; d. negative controls, Quantiferon negative, BCG vaccinated.

FIG. 2. Analysis of IFN-gamma production as TAWITAVVPGLMV (SEQ ID NOT 24) peptide response from 4 tested subject groups.

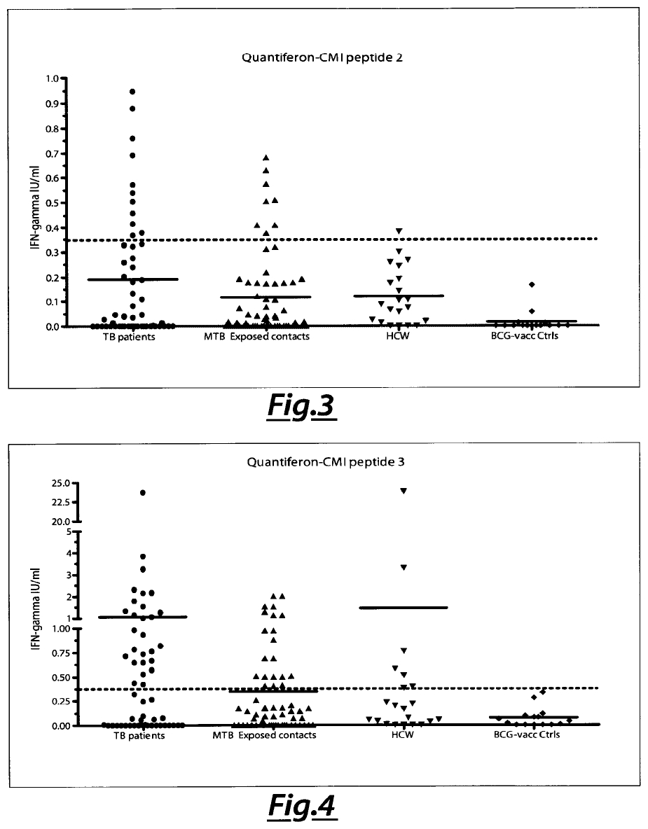

FIG. 3. Analysis of IFN-gamma production as AVIVRSELLTQYL (SEQ ID NO 22) peptide response from 4 tested subject groups.

FIG. 4. Analysis of IFN-gamma production as GSVRQLPSVLKPPLITLRTLTLSG (SEQ ID NO 71) peptide response from 4 tested subject groups.

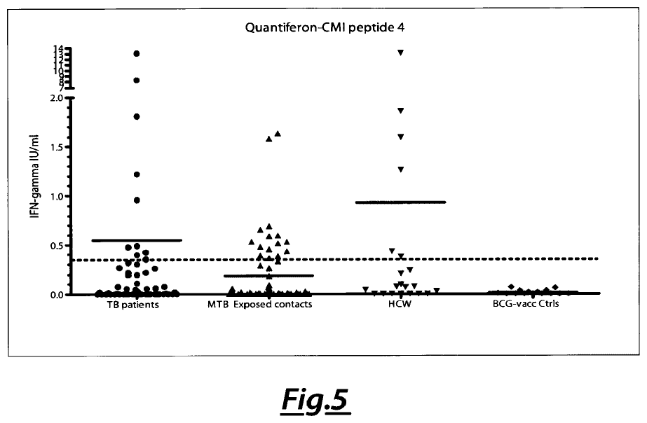

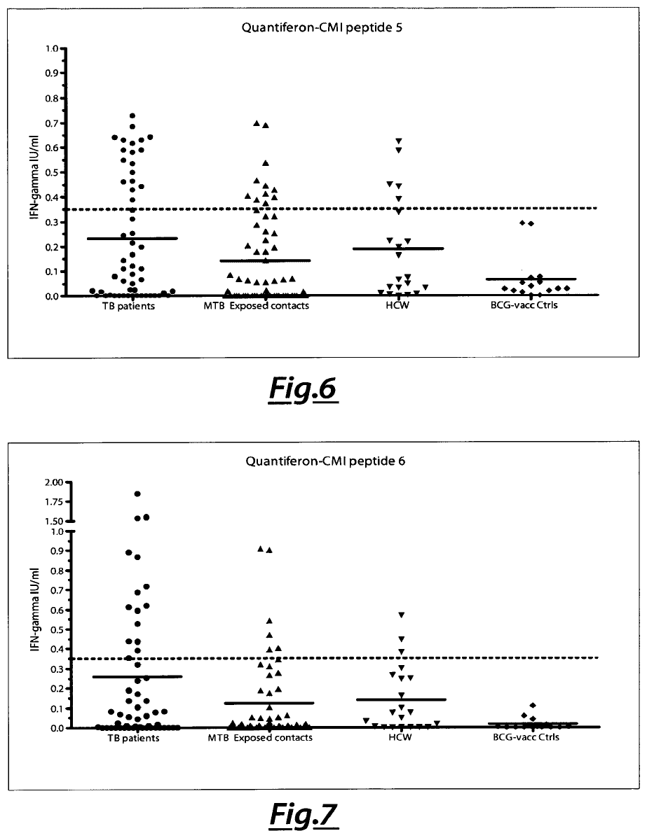

FIG. 5. Analysis of IFN-gamma production as RPVRRVLLFVVPSSGPAP (SEQ ID NO 70) peptide response from 4 tested subject groups.

FIG. 6. Analysis of IFN-gamma production as GEIIFISGRLNGaa (SEQ ID NO 13) peptide response of tested samples.

FIG. 7. Analysis of IFN-gamma production as ELMARAAVLGSAH (SEQ ID NO 21) peptide response of tested samples.

FIG. 8. Analysis of IFN-gamma production as ESAT6 (QQWNFAGIEAAASAIQGNVTSIHSL—SEQ ID NO:84) response of tested samples.

FIG. 9. Frequency of positive tests using only SEQ ID NO:24,21,71,70,13,22 six peptides.

FIG. 10. Sensitivity increase of Quantiferon TB-plus commercial used test after SEQ ID NO: 24,21,70,71,13,22 peptide addition thereto.

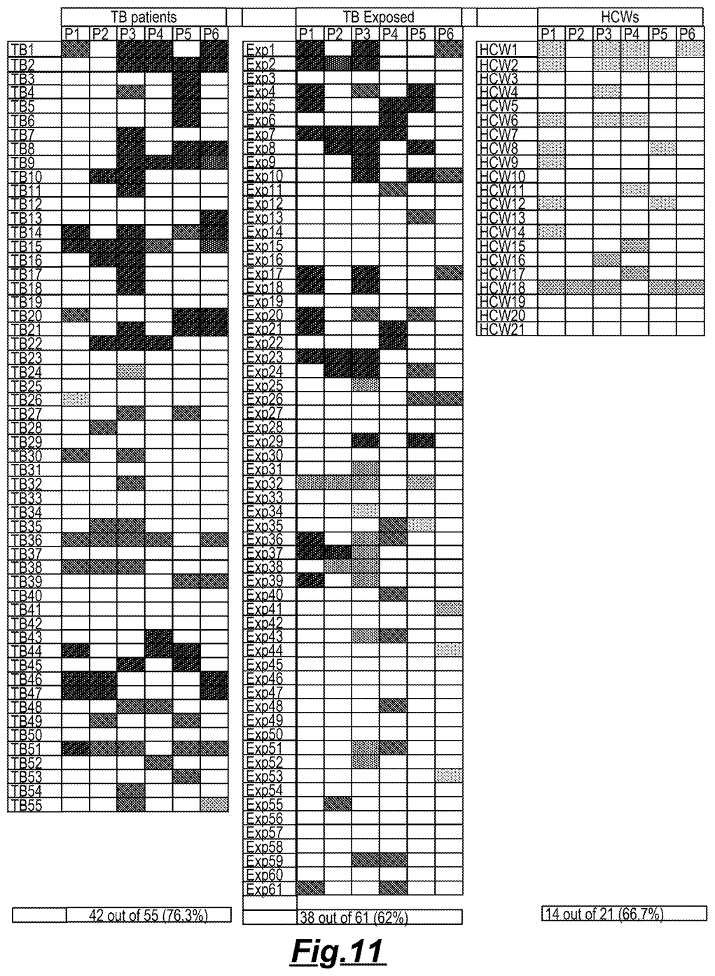

FIG. 11. Combination use of 1-6 peptides, which allow in panel a 76.3%, 62% and 66.7%, compared to 55%, 33% and 45.5% sensitivity obtained using RD1 (ESAT6) multiepitope peptide, for TB+ subjects, PPD+ esposed contacts and HCWs, respectively, to be obtained.

### EXAMPLE 1

### Identification of M. tuberculosis Expressed Proteins in Human Infected Macrophages from Both Vitro and Ex Vivo Analyzed Biological Samples

### Materials and Methods

### ELISPOT Immunodiagnostic Test

The global procedure for test carrying out demands for: 96 well plates (MAIPS45, Millipore, Sunnyvale, CA, USA); primary antibody (IFN-gamma coating monoclonal, M-700A, Pierce-Endogen Inc, Rockford, USA); biotynilated antibody (M-701B, Pierce-Endogen Inc); streptavidin-HRP (Pierce-Endogen); substrate (AEC Staining kit, Sigma); stimulus (peptides, PHA and other antigens) at ready to use concentration.

ELISPOT procedure is carried out according to the following step:

Coating: treatment of a 96 well plate using 5 μg/mL sterile phosphate buffer (PBS) solution of primary antibody delivered at 100 μL/well; coat the plate and incubate at 4° C. for 20 hours; wash the plate 4 times using 200 μL/well of sterile PBS, at final washing eliminate excess liquid by tapping plate on adsorbent paper.

Blocking: addition of 200 μL/well of “blocking solution” [sterile PBS containing 10% fetal calf serum (FCS)], in order to prevent not specific protein binding; plate incubation for 2 hours at room temp.; “blocking solution” suction.

### Cell Preparation and Incubation

1. isolation of mononucleated cells (PBMC) from venous blood (7 ml with EDTA) by density gradient centrifugation (Ficoll-Hypaque, Pharmacia; Uppsala; Swedewn), using a quick method based on the use of filtering tubes for leucocytes separation (LeucoSep™, ARNIKA, Milan). After two washings with PBS (phosphate buffered saline) 1×, the pellet is resuspended in complete medium (RPMI 1640 containing HEPES 25 mM, 10% v/v FCS, 2 mM L-Glutamine, 10 U/mL penicillin/streptomycin)) in order to have 2×10⁵ cells in 100 μL.
2. add 100 μL/well of cellular suspension together with 100 μL of different stimula;
3. incubate the plate for 40 hours at 37° C. in incubator at 5% CO₂;
4. remove the cells;
5. wash the plate 4 times with 200 μL/well of PBS and 4 times with 200 μL/well of “Wash buffer” [PBS/0.05% Tween 20 (Sigma)];
6. at the final washing eliminate the excess liquid by tapping the plate on absorbent paper.
   Incubation with Biotinilated Antibody

100 μl/well of biotinilated antibody diluted in PBS/4% bovine serum albumin (fraction V, Sigma) at concentration of 1 μg/ml have been delivered. Then the plate has been incubated for 100 minutes at 37° C. in incubator at 5% CO₂; the plate has been washed 4 times with “Wash buffer”. At final washing excess liquid has been eliminated by tapping plate on adsorbent paper.

### Detection

For the detection 100 μl/well of “Streptavidin-HRP” 1:1000 diluted in “Wash buffer”. The plate has been incubated for 30 minutes at room temp. in the dark, then washed 4 times with “Wash buffer”. At final washing excess liquid has been eliminated by tapping plate on adsorbent paper. 100 μL/well of substrate have been delivered. In parallel as a control for enzyme-substrate reaction occurrence 100 μl of as prepared substrate with 100 μl of “diluted Streptavidin-HRP” has been incubated for few minutes. If the reaction is successful the substrate turns out from light brown to pink colour.

Finally, the plate has been incubated for 10-20 minutes at room temp. in the dark. The substrate has been discarded, the plate washed with water eliminating excess and air dried for 20 hours.

ELISA tests for the identification of human and animal IFN-gamma in whole blood samples stimulated with selected peptides and proteins (CMI test protocol).

### Results

The authors have identified the group of M. tuberculosis expressed proteins in human infected macrophages from both in vitro and ex vivo analyzed biological samples. M. tuberculosis gene expressions in three different growth environments have been compared: synthetic medium (Sauton's), monocyte-derived-human macrophage (MDM) infected in culture with M. tuberculosis, alveolar macrophage (AM) from bronchoalveolar lavage (BAL) samples of pulmonary TB affected patients before the antibiotic therapy.

From thus obtained 9 groups of genes, first 100 proteins, according to a combination criteria (immunogenicity, tubercular Complex specificity, etc), have been selected. From these 100 proteins, again a 30 protein group has been selected, for which a positive response in immunological tests on TB patient whole blood had been obtained.

TABLE 1

| | | Amino | |
|---|---|---|---|
| | | acid | |
| | | sequence | |
| | In vivo | ID | |
| Rv | modulation | (UNIPROT) | Already tested peptides |
| Rv0023 | B, E | P67704 | EMWDIRNRGVIPAGALPRVR (SEQ ID NO: 1) |
| Rv0182c | E | O07426 | AKFRSVRVVVITGSVTAAPVRVSETLRRLI (SEQ |
| | | | ID NO: 2), ESVRLAFVAALQH (SEQ ID NO: 3) |
| Rv0290 | D | O86362 | GLLITIRSPRSGIA (SEQ ID NO: 4), |
| | | | AQLLWQLPLLSIG (SEQ ID NO: 5) |
| Rv0601c | C, E | O07777 | ADLVRELVTILPIVLVIAAVAAYLLSR |
| | | | (SEQ ID NO: 6) |
| | | | AAYLLSRAALRPVDRIRAAA (SEQ ID NO: 7) |
| | | | TTLNTMLTRLQRALAHEQQF (SEQ ID NO: 8) |
| | | | DLFVSIDPDHLRRILTAVLDN (SEQ ID NO: 9) |
| | | | SGLGLAIVAALTTTHGG (SEQ ID NO: 10) |
| Rv0647c | E | P96936 | GRLPRKGPWQQKVIKELPQ (SEQ ID NO: 11), |
| | | | GKIVVLMGAVGTMKPETQAA (SEQ ID NO: 12) |
| Rv0724A | B | Q79FX1 | GEIIFISGRLNGaa (SEQ ID NO: 13) |
| Rv0890c | C, E | Q10550 | ARVRSMSPLEIAD (SEQ ID NO: 14), |
| | | | EQILFRRLAPFVGGF (SEQ ID NO: 15), |
| | | | AALVRALTACGCSS (SEQ ID NO: 16), |
| | | | DKWTLCQILYWRGVGTCISGD (SEQ ID NO: 17), |
| | | | TKVLGLYTQAQVLAYCG (SEQ ID NO: 18), |
| | | | DQVTMHQVLMAQLALAGG(SEQ ID NO: 19), |
| | | | EGVRLLGAAAALRQQTRQVRFK (SEQ ID |
| | | | NO: 20) |
| Rv1251c | C, E | O50466 | ELMARAAVLGSAH (SEQ ID NO: 21), |
| | | | AVIVRSELLTQYL (SEQ ID NO: 22) |
| Rv1398c | D | P64835 | GTLRHLDPPVRRSGGREQHL (SEQ ID NO: 23) |
| Rv1478 | E | O53169 | TAWITAVVPGLMV (SEQ ID NO: 24) |
| Rv1497 | C, E | P71778 | APMVFSATKGMTA (SEQ ID NO: 25), |
| | | | TCAMRRLAHRFSGG(SEQ ID NO: 26) |
| Rv1575 | C, E | O06615 | SVVRRKQTLLSAQ (SEQ ID NO: 27) |
| Rv1578c | E | O06612 | GVVHRNPAVTVAE (SEQ ID NO: 28) |
| Rv1899c | D | O07733 | PGVVATHAVRTLGTTGSRAIGL (SEQ ID NO: 29), |
| | | | PQWRRARVRLCGRWWRRSNTTRGAS (SEQ ID NO: 30), |
| | | | ARLMVGAVRRHRPGSLQR (SEQ ID NO: 31) |
| Rv2137c | D | O06238 | aaMRNMKSTSHE (SEQ ID NO: 32) |
| Rv2333c | E | P71879 | QTIVMLWTAAVGCA (SEQ ID NO: 33), |
| | | | LCMLMLGLLMLIFSEHRSS(SEQ ID NO: 34), |
| | | | SALVLVGLGLCGSGVALCLT (SEQ ID NO: 35) |
| Rv2548 | A, E | P95005 | SELVRFELLAGVRESE (SEQ ID NO: 36), |
| | | | VDYLIAATAIVVDA (SEQ ID NO: 37) |
| Rv2557 | A, E | P65003 | QGIEYYKSSVLPQIE (SEQ ID NO: 38), |
| | | | EGWIVYARSTTIQAQS (SEQ ID NO: 39), |
| | | | TRRMYSNYGF (SEQ ID NO: 40) |
| Rv2816c | A | P71637 | FGYRVQESAFEAMLTKGQLAKLV (SEQ ID NO: 41), |
| | | | DNIRIYKIRGVAAVTFYGRGRLVSAE (SEQ ID NO: 42) |
| Rv2990 | E | O53239 | RSYILRAGISSLFRYIEGVHGER (SEQ ID NO: 43), |
| | | | SAMRPQDRLLVGNWVDDSLL (SEQ ID NO: 44), |
| | | | LYLVGLEPYVQFE (SEQ ID NO: 45), |
| | | | AGFRILEARRFPI (SEQ ID NO: 46), |
| | | | IRYRARYVNGQLNMCLARI (SEQ ID NO: 47) |
| Rv3094c | E | O05773 | ALLVAYLPARSREEMF (SEQ ID NO: 48), |
| | | | NRLRLAATHAVRT(SEQ ID NO: 49), |
| | | | APLQRRFRDAFTATAHFQVNE(SEQ ID NO: 50), |
| | | | SRELPGRVLLDQPADVSM (SEQ ID NO: 51) |
| Rv3107c | A, E | O05784 | EPVVTVDVTAMSAVLEID (SEQ ID NO: 52) |
| Rv3188 | C, E | O53334 | AVIQVSDRAVRGWRTGDIRPERY (SEQ ID NO: 53) |
| Rv3239c | C, E | O05884 | PDLRGALLLAVTLGLVT (SEQ ID NO: 54), |
| | | | PDWGWLSVATVGSFLA(SEQ ID NO: 55) |
| | | | GAVLGVAVMVILIGKPEHGTA (SEQ ID NO: 56), |
| | | | AAICFIAVAVAAAVL(SEQ ID NO: 57), |
| | | | TKLVRLTKAQFDEIA(SEQ ID NO: 58), |
| | | | ADLVLAGPAASREH(SEQ ID NO: 59), |
| | | | YAYEYFIRHNPLSDYA(SEQ ID NO: 60), |
| | | | FPVRGLVRGRRTLTLLEA (SEQ ID NO: 61) |
| Rv3296 | A, E | P96901 | EVLRILRRRSLAALRA (SEQ ID NO: 62), |
| | | | RVILHSPYGLRVHGPLAL (SEQ ID NO: 63) |
| Rv3425 | E | Q50703 | AAWVINGLANAYNDT (SEQ ID NO: 64), |
| | | | DQYRARNVAVMNAYVSWTRSALSDLPR (SEQ ID NO: 65), |
| | | | SDLLADAVERYLQWLSKSSSQLKHA (SEQ ID NO: 66) |
| Rv3446c | C, E | O06263 | GPVVVHPSWWSAA (SEQ ID NO: 67), |
| | | | ITAVVLIDVPSTVAGA (SEQ ID NO: 68), |
| | | | AAVVRHGATTLQRP (SEQ ID NO: 69) |
| Rv3479 | C, E | O06342 | RPVRRVLLFVVPSSGPAP (SEQ ID NO: 70), |
| | | | GSVRQLPSVLKPPLITLRTLTLSG (SEQ ID NO: 71), |
| | | | SALLRRLSTCPPES (SEQ ID N: 87) |
| Rv3482c | E | O06345 | GAVLRLVVRFAEPLPPSP (SEQ ID NO: 72), |
| | | | AGYLLTYTIANNGKEFAEL (SEQ ID NO: 73) |
| Rv3780 | D | P65091 | aaVRKRMVIGLSTGSDDD (SEQ ID NO: 74) |
| Cons FS | | | ALLLRDVLQWKSAEVADAIG (SEQ ID NO: 75) |
| | | | NSLLQRARSQLQTVRPSAADRLSAA (SEQ ID NO: 76) |
| Cons | | | MSWVMVSPELVVAAAADLAG (SEQ ID NO: 77) |
| PE_PGRS | | | AAFYAQFVQALTSGGAY (SEQ ID NO: 78) |
| Cons REG | | | ALLVRMPTSLPAVA (SEQ ID NO: 79) |
| Cons CW | | | SRLRTHVRPDAPLVPLALRVDGLRSRW (SEQ ID NO: 80) |
| | | | AAVLTMLGVAGYGW (SEQ ID NO: 81) |
| | | | GLFMIFLDALIVNVALPDIQR (SEQ ID NO: 82) |
| | | | SWVVASYSLGMAVFIMSAGTLADLL (SEQ ID NO: 83) |

Legend of modulation:
A: up-regulated in AM vs MDM;
B: always expressed in AM and MDM;
C: up-regulated in MDM vs AM;
D: up-regulated in Sauton vs MDM and/or AM;
E: up-regulated in MDM and/or AM vs Sauton's

After an ulterior selection initially 43 peptides from 4 groups of subjects: Pulmonary TB before antibiotic therapy (n=13), recently exposed healthy contacts (TB patient relations) PPD+ (n=8); long TB patient exposed healthy contacts (professional exposure of hospital workers) PPD+ (n=5); BCG vaccinated negative controls, PPD− (n=4), have been designed, synthesized and tested.

Then 6 most sensible and specific peptides have been selected (see table 2) and the study has been repeated using an extended subject sample (see tables 3-4 and FIGS. 1-7).

Table 2 reports MTB selected genes, peptides selected for T CD4+ cell assay and corresponding identification number thereof, respectively.

TABLE 2

| | | | In vivo |
|---|---|---|---|
| No | Peptide | Gene | modulation |
| SEQ ID | TAWITAV | Consensus | Induced in AM and |
| NO: 24 | VPGLMV | VIR(Rv1478) | MDM vs Sauton's |
| SEQ ID | AVIVRSE | Rv1251c | Induced in MDM |
| NO: 22 | LLTQYL | | vs Sauton's |
| SEQ ID | GSVRQLPSVLKP | Rv3479 | Induced in MDM |
| NO: 71 | PLITLRTLTLSG | | vs Sauton's |
| SEQ ID | RPVRRVLL | Rv3479 | Induced in MDM |
| NO: 70 | FVVPSSGPAP | | vs Sauton's |
| SEQ ID | GEIIFISGRL | Rv0724A | Expressed in AM |
| NO: 13 | NGaa | | and MDM |
| SEQ ID | ELMARAAVLG | Rv1251c | Induced in MDM |
| NO: 21 | SAH | | vs Sauton's |

TABLE 3

| | Microbiologically | Recently | | |
|---|---|---|---|---|
| | checked TB | MTB | | BCG- |
| | patients (before | exposed | Sanitary | vaccinated |
| | therapy start) | subjects | workers | controls |
| Examined | 58 | 63 | 21 | 15 |
| subject number | | | | |
| Anergic subjects | 3/58 | 2/63 | 0/21 | 0/15 |
| (mitogene low | | | | |
| response or no | | | | |
| response to all | | | | |
| stimula) | | | | |
| Quantiferon | 41/55 | 25/61 | 11/21 | 3/15 |
| TB-Gold in-Tube | | | | |
| PPD | 55/55 | 61/61 | 21/21 | 15/15 |
| ESAT-6 control | 28/55 | 17/61 | 7/21 | 0/15 |
| peptide | | | | |
| Peptide #1 | 12/55 | 14/61 | 6/21 | 0/15 |
| (Seq ID N°:24) | | | | |
| Peptide #2 | 11/55 | 8/61 | 1/21 | 0/15 |
| (Seq ID N°:22) | | | | |
| Peptide #3 | 27/55 | 20/61 | 7/21 | 0/15 |
| (Seq ID N°:71) | | | | |
| Peptide #4 | 10/55 | 13/61 | 6/21 | 0/15 |
| (Seq ID N°:70) | | | | |
| Peptide #5 | 18/55 | 11/61 | 5/21 | 0/15 |
| (Seq ID N°:13) | | | | |
| Peptide #6 | 14/55 | 6/61 | 3/21 | 0/15 |
| (Seq ID N°:21) | | | | |

TABLE 4

| | Microbiologically | Recently | | |
|---|---|---|---|---|
| | checked TB | MTB | | BCG- |
| | patients (before | exposed | Sanitary | vaccinated |
| | therapy start) | subjects | workers | controls |
| Examined | 58 | 63 | 21 | 15 |
| subject number | | | | |
| Anergic subjects | 3/58 | 2/63 | 0/21 | 0/15 |
| (mitogene low | | | | |
| response or no | | | | |
| response to all | | | | |
| stimula) | | | | |
| Quantiferon | 41/55 | 25/61 | 11/21 | 3/15 |
| TB-Gold in-Tube | | | | |
| Panel 1-6 | 40/55 | 30/61 | 11/21 | 0/15 |
| peptides | | | | |
| QFT Gold + | 49/55 | 34/61 | 13/21 | 3/15 |
| panel 1-6 | | | | |
| peptides | | | | |

TABLE 5

| | Microbiologically | Recently | | |
|---|---|---|---|---|
| | checked TB | MTB | | BCG- |
| | patients (before | exposed | Sanitary | vaccinated |
| | therapy start) | subjects | workers | controls |
| Examined | 38 | 32 | 10 | 10 |
| subject number | | | | |
| Anergic | 3 | 1 | 0 | 0 |
| 1-6 peptides | 22/35 | 10/31 | 4/10 | 1/10 |
| (in pool) | | | | |
| QFT Gold | 25/35 | 11/31 | 4/10 | 2/10 |
| QFT Gold + 1-6 | 29/35 | 13/31 | 5/10 | 2/10 |
| peptides in | | | | |
| same well | | | | |

Results obtained with said six peptides and peptide belonging to ESAT6 protein, i.e. a highly immunogenic protein occurring in both above mentioned commercial kits, have been compared.

MTB genes observed as induced, both in course of human macrophage infection and/or in alveolar macrophage samples from active pulmonary TB patients, are indicated in the following list:
Genes always expressed during intracellular replication in MDM and AM: Rv0724A.
Genes induced in AM and/or MDM vs Sauton's medium culture: Rv1251c, Rv1478 and Rv3479.

Two groups of MTB genes share probable role in the survival inside human host cell (both primary macrophages from healthy in vitro infected donors and TB patient alveolar macrophages) resulting in design thereof as MTB intracellular survival bio-markers, whereas MTB virulence definition is just based on the pathogen ability to invade, survive and replicate within the host cell.

Moreover, the authors of the present invention have designed peptides of some gene groups belonging to same metabolic category, in order to find “consensus” protein sequences for said categories. The research is based on the assumption that functional domains of similar function exploiting proteins occurring in various bacterial species, are conserved. In order to find these conserved motifs the sequence multiple alignment (PSSM), using PSI-BLAST (Position Specific Iterated Basic Local Alignment Search Tool, http://www.ncbi.nlm.nih.gov/BLAST), is generated. After the sequences with higher similarity grades with the inserted sequence has been detected, it is possible to select the proteins suitable to contribute to the generation of the profile used for the successive data bank search; in this way the number of the sequences contributing to the generation of the profile is different for the different sequence positions.

A multi-alignment allows structurally and functionally important, because extremely conserved, residues to be detected and said residues as a whole will constitute the “consensus” sequence or sequences for each MTB protein functional group.

Therefore proteins (induced or repressed in human macrophage) of metabolic functional groups (for example regulatory proteins, lipid metabolism involved proteins, etc.) detected as “modulated” by M. tuberculosis during infection course, have been analyzed for search of conserved sequences. Using PSI-BLAST sequence various multiple alignments, from which we have reached the best “consensus” sequences for the peptide synthesis, have been obtained.

The peptides derived from selected proteins have been synthesized and used for the detection and quantification of MTB specific T CD4+ lymphocytes using detection system for IFN-γ producing cells both with ELISPOT technique and with TB diagnosis high sensitivity ELISA assay, Quantiferon TB-Plus and Quantiferon CMI. This technique allows the frequency of T cells producing a determined cytokine (for example, IFN-γ) as a response to a specific antigenic stimulus suggesting that the immune system of treated subjects has been able to evoke an immune response towards said peptides when infectious agent (MTB) encoding for the same occurs, to be quantified. The second technique allows total IFN-gamma production resulting from specific T lymphocytes as a response to selected antigens, to be quantified.

Although this test does not represent the evidence of ability thereof to induce protection from MTB infection, the occurred detection of the presence of lymphocytes recognizing these peptides specifically and differently in MTB infected subjects or with active tuberculosis, is an index of their immunogenicity, indispensable minimal characteristic in order a vaccine and a diagnostic test to be proved effective. Further these peptides, alone or in addition to other mycobacterial antigens, allow a sensitive and specific test for TB diagnosis to be provided and the sensitivity of commercial test, i.e. current reference gold standard for the tubercular diagnosis, to be enhanced (FIG. 10). When panel evaluated and directly tested within same well, 6 selected peptides allow Quantiferon TB gold in-Tube response from 75% to 89% (+14%) and from 71% to 83% (+13%), respectively, for subjects with active TB, to be enhanced, without decreasing the assay specificity.

### BIBLIOGRAPHY

1. EW Koneman, SD Allen, WM Janda, P. Schreckenberger, WC Winn. Color Atlas and Textbook of Diagnostic Microbiology. 5ª edizione. Lippincot, 1997
2. Menzies D, Pai M, Comstock G. Ann Intern Med. 2007 Mar. 6; 146(5):340-54. Meta-analysis: new tests for the diagnosis of latent tuberculosis infection: areas of uncertainty and recommendations for research
3. Bellete B, Coberly J, Barnes GL, Ko C, Chaisson RE, Comstock GW, Bishai WR. Clin Infect Dis. 2002 34:1449-56.
4. Pai M, Gokhale K, Joshi R, Dogra S, Kalantri S, Mendiratta DK, Narang P, Daley CL, Granich RM, Mazurek GH, Reingold AL, Riley LW, Colford JM Jr. JAMA. 2005 293:2746-55.
5. Lalvani A, Pathan AA, Durkan H, Wilkinson KA, Whelan A, Deeks JJ, Reece WH, Latif M, Pasvol G, Hill AV. Lancet. 2001; 357: 2017-21.
6. Centers for Disease Control and Prevention. MMWR Recomm Rep. 1997 Sep. 5; 46(RR-15):1-10.
7. Ulrichs T, Munk ME, Mollenkopf H, et al. Eur J Immunol 1998; 28:3949-3958.
8. Ravn P, Demissie A, Eguale T, et al. J Infect Dis 1999; 179:637-645.
9. Doherty TM, Demissie A, Olobo J, et al. J Clin Microbiol 2002; 40:704-706.
10. Lalvani A, Pathan AA, Durkan H, et al. Lancet 2001; 357:2017-2021.
11. Lalvani A, Nagvenkar P, Udwadia Z, et al. J Infect Dis 2001; 183:469-477.
12. Chapman AL, Munkanta M, Wilkinson KA, et al. AIDS 2002; 16:2285-2293.
13. Lalvani A, Pathan AA, McShane H, et al. Am J Respir Crit Care Med 2001; 163:824-828.
14. Pathan AA, Wilkinson KA, Klenerman P, et al. J Immunol 2001; 167:5217-5225.
15. LinksLawn SD, Bangani N, Vogt M, Bekker LG, Badri M, Ntobongwana M, Dockrell HM, Wilkinson RJ, Wood R. Utility of interferon-γ ELISPOT assay responses in highly tuberculosis-exposed patients with advanced HIV infection in South Africa.BMC Infect Dis. 2007 Aug. 28; 7:99
16. De Groot AS, Bosma A, Chinai N, Frost J, Jesdale BM, Gonzalez MA, Martin W, Saint-Aubin C. Vaccine 2001, 19:4385-4395.
17. Brander C, Goulder PJR. Edited by Korber BTM, Brander C, Haynes B, Koup RA, Kuiken C, Moore J, Walker B, Watkins D. Los Alamos National Laboratory, Los Alamos, N.Mex. 2000; I1-I3.

1. A method for detecting a Mycobacterium species infection in a test subject, comprising:
(a) incubating in vitro a blood sample or a lavage sample that comprises lymphocytes from the test subject with at least one isolated protein or peptide that comprises at least one Mycobacterium T-cell epitope, said isolated protein or peptide being selected from:
(i) SEQ ID NO:1 (Rv0023), SEQ ID NOS:2-3 (Rv0182c), SEQ ID NOS: 4-5 (Rv0290), SEQ ID NOS:6-10 (Rv0601c), SEQ ID NOS:11-12 (Rv0647c), SEQ ID NO:13 (Rv0724A), SEQ ID NOS:14-20 (Rv0890c), SEQ ID NOS:21-22 (Rv1251c), SEQ ID NO:23 (Rv1398c), SEQ ID NO:24 (Rv1478), SEQ ID NOS:25-26 (Rv1497), SEQ ID NO:27 (Rv1575), SEQ ID NO:28 (Rv1578c), SEQ ID NOS:29-31 (Rv1899c), SEQ ID NO:32 (Rv2137c), SEQ ID NOS:33-35 (Rv2333c), SEQ ID NOS:36-37 (Rv2548), SEQ ID NOS:38-40 (Rv2557), SEQ ID NOS:41-42 (Rv2816c), SEQ ID NOS:43-47 (Rv2990), SEQ ID NOS:48-51 (Rv3094c), SEQ ID NO:52 (Rv3107c), SEQ ID NO:53 (Rv3188), SEQ ID NOS:54-61 (Rv3239c), SEQ ID NOS:62-63 (Rv3296), SEQ ID NOS:64-66 (Rv3425), SEQ ID NOS:67-69 (Rv3446c), SEQ ID NOS:70, 71, and 87 (Rv3479), SEQ ID NOS:72-73 (Rv3482c), and SEQ ID NO:74 (Rv3780);
(ii) a homolog of any one of the proteins or peptides of (i) having an amino acid sequence with at least 80% similarity to said protein or peptide of (i) after optimal alignment and comprising at least one Mycobacterium T-cell epitope; and
(iii) a peptide fragment of the protein or peptide as defined in (i) or (ii), said peptide fragment having a T-cell epitope, or a chemical analog thereof,
wherein said step of incubating is for a time and under conditions sufficient to stimulate the lymphocytes to produce an effector molecule; and
(b) detecting a test level of the effector molecule, wherein detection of a test level that is higher than a control level that is detected following incubation of control lymphocytes from a healthy control subject, indicates that the test subject is infected with, or has been previously exposed to, the Mycobacterium species.

2. The method of claim 1 wherein the Mycobacterium species is selected from M. tuberculosis, M. bovis, M. bovis BCG, M. africanum, M. canetti, M. caprae, M. microti, M. pinnipedii, M. avium, M. avium paratuberculosis, M. avium silvaticum, M. avium “hominissuis”, M. colombiense, M. asiaticum, M. gordonae, M. gastri, M. kansasii, M. hiberniae, M. nonchromogenicum, M. terrae, M. triviale, M. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. simiae, M. branderi, M. cookii, M. celatum, M. bohemicum, M. haemophilus. malmoense, M. szulgai, M. leprae, M. lepraemurium, M. lepromatosis, M. africanum, M. botniense, M. chimaera, M. conspicuum, M. doricum, M. farcinogenes, M. heckeshornense,. intracellulare, M. lacus, M. marinum, M. monacense, M. montefiorense, M. murale, M. nebraskense, M. saskatchewanense, M. scrofulaceum, M. shimoidei, M. tusciae, M. xenopi, M. intermedium, M. abscessus, M. chelonae, M. bolletii, M. fortuitum, M. fortuitum subsp. acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diernhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodii, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, M. seoulense and M. tokaiense.

3. The method of claim 1 wherein one or more of:
(a) the Mycobacterium species is Mycobacterium tuberculosis,
(b) either the subject is a human or the subject is a non-human animal, and
(c) one of:
(1) the at least one isolated protein or peptide that comprises at least one Mycobacterium T-cell epitope is an Rv0890c peptide that is selected from SEQ ID NOS: 14-19 and 20, or an Rv2333c peptide that is selected from SEQ ID NOS: 33, 34 and 35, or an Rv2548 peptide that is selected from SEQ ID NOS: 36 and 37,
(2) in the step of incubating there is present one or more Mycobacterium protein, or a peptide fragment thereof or a chemical analog derived therefrom, wherein the one or more Mycobacterium protein is selected from ESAT6, CFP10, TB7.7 and PPD, and wherein the detected test level of the effector molecule is greater than a detectable effector molecule level that can be produced by said lymphocytes incubated only with said one or more Mycobacterium protein selected from ESAT6, CFP10, TB7.7 and PPD, or
(3) both (1) and (2).

4. A method for detecting a Mycobacterium species infection in a test subject, comprising:
(a) incubating in vitro a biological sample that comprises lymphocytes from the test subject with at least one protein or peptide that is selected from:
(i) an isolated protein or peptide that comprises at least one T-cell epitope and that comprises an amino acid sequence from a Mycobacterium species and is selected from SEQ ID NO:1 (Rv0023), SEQ ID NOS:2-3 (Rv0182c), SEQ ID NOS: 4-5 (Rv0290), SEQ ID NOS:6-10 (Rv0601c), SEQ ID NOS:11-12 (Rv0647c), SEQ ID NO:13 (Rv0724A), SEQ ID NOS:14-20 (Rv0890c), SEQ ID NOS:21-22 (Rv1251c), SEQ ID NO:23 (Rv1398c), SEQ ID NO:24 (Rv1478), SEQ ID NOS:25-26 (Rv1497), SEQ ID NO:27 (Rv1575), SEQ ID NO:28 (Rv1578c), SEQ ID NOS:29-31 (Rv1899c), SEQ ID NO:32(Rv2137c), SEQ ID NOS:33-35 (Rv2333c), SEQ ID NOS:36-37 (Rv2548), SEQ ID NOS:38-40 (Rv2557), SEQ ID NOS:41-42 (Rv2816c), SEQ ID NOS:43-47 (Rv2990), SEQ ID NOS:48-51 (Rv3094c), SEQ ID NO:52 (Rv3107c), SEQ ID NO: 53 (Rv3188), SEQ ID NOS:54-61 (Rv3239c), SEQ ID NOS:62-63 (Rv3296), SEQ ID NOS:64-66 (Rv3425), SEQ ID NOS:67-69 (Rv3446c), SEQ ID NOS:70, 71, and 87 (Rv3479), SEQ ID NOS:72-73 (Rv3482c), and SEQ ID NO:74 (Rv3780);
(ii) a homolog of the protein or peptide as defined in (i) having an amino acid sequence with at least 80% similarity to said protein or peptide after optimal alignment and comprising at least one Mycobacterium T-cell epitope, and
(iii) a peptide fragment of the protein or peptide as defined in (i) or (ii) having a T-cell epitope, or a chemical analog thereof, or
(b) incubating in vitro a biological sample that comprises lymphocytes from the test subject with at least one protein or peptide of (a)(i), (a)(ii) or (a)(iii) and further with a Mycobacterium protein, or a fragment thereof or a chemical analog thereof, said Mycobacterium protein being selected from:
(a) ESAT,
(b) CFP10,
(c) TB7.7, and
(d) PPD,
wherein said step (a) or (b) of incubating is for a time and under conditions sufficient to stimulate the lymphocytes to produce an effector molecule; and
(c) detecting a test level of the effector molecule,
wherein following step (b) the detected test level of the effector molecule is greater than a detectable effector molecule level that can be produced by said lymphocytes incubated only with said one or more Mycobacterium protein selected from ESAT6, CFP10, TB7.7 and PPD,
and wherein following either step (a) or step (b), detection of a test level that is higher than a control level that is detected following incubation of control lymphocytes from a healthy control subject, indicates that the test subject is infected with, or has been previously exposed to, the Mycobacterium species.

5. The method of claim 4 wherein the Mycobacterium species is selected from M. tuberculosis, M. bovis, M. bovis BCG, M. africanum, M. canetti, M. caprae, M. microti, M. pinnipedii, M. avium, M. avium paratuberculosis, M. avium silvaticum, M. avium “hominissuis”, M. colombiense, M. asiaticum, M. gordonae, M. gastri, M. kansasii, M. hiberniae, M. nonchromogenicum, M. terrae, M. triviale, M. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. simiae, M. branderi, M. cookii, M. celatum, M. bohemicum, M. haemophilus. malmoense, M. szulgai, M. leprae, M. lepraemurium, M. lepromatosis, M. africanum, M. botniense, M. chimaera, M. conspicuum, M. doricum, M. farcinogenes, M. heckeshornense, M. intracellulare, M. lacus, M. marinum, M. monacense, M. montefiorense, M. murale, M. nebraskense, M. saskatchewanense, M. scrofulaceum, M. shimoidei, M. tusciae, M. xenopi, M. intermedium, M. abscessus, M. chelonae, M. bolletii, M. fortuitum, M. fortuitum subsp. acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diernhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodii, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, seoulense and M. tokaiense.

6. The method of claim 4 wherein the at least one isolated protein or peptide that comprises at least one Mycobacterium T-cell epitope is an Rv0890c peptide that is selected from SEQ ID NOS: 14-19 and 20, or an Rv2333c peptide that is selected from SEQ ID NOS: 33, 34 and 35, or an Rv2548 peptide that is selected from SEQ ID NOS: 36 and 37.

7. An isolated protein selected from:
(a) an isolated protein that comprises an amino acid sequence from a Mycobacterium species and that is selected from SEQ ID NO:1 (Rv0023), SEQ ID NOS:2-3 (Rv0182c), SEQ ID NOS: 4-5 (Rv0290), SEQ ID NOS:6-10 (Rv0601c), SEQ ID NOS:11-12 (Rv0647c), SEQ ID NO:13 (Rv0724A), SEQ ID NOS:14-20 (Rv0890c), SEQ ID NOS:21-22 (Rv1251c), SEQ ID NO:23 (Rv1398c), SEQ ID NO:24 (Rv1478), SEQ ID NOS:25-26 (Rv1497), SEQ ID NO:27 (Rv1575), SEQ ID NO:28 (Rv1578c), SEQ ID NOS:29-31 (Rv1899c), SEQ ID NO:32 (Rv2137c), SEQ ID NOS:33-35 (Rv2333c), SEQ ID NOS:36-37 (Rv2548), SEQ ID NOS:38-40 (Rv2557), SEQ ID NOS:41-42 (Rv2816c), SEQ ID NOS:43-47 (Rv2990), SEQ ID NOS:48-51 (Rv3094c), SEQ ID NO:52 (Rv3107c), SEQ ID NO:53 (Rv3188), SEQ ID NOS:54-61 (Rv3239c), SEQ ID NOS:62-63 (Rv3296), SEQ ID NOS:64-66 (Rv3425), SEQ ID NOS:67-69 (Rv3446c), SEQ ID NOS:70, 71, and 87 (Rv3479), SEQ ID NOS:72-73 (Rv3482c), and SEQ ID NO:74 (Rv3780); said isolated protein comprising a mycobacterial epitope that is recognized by T-cells, and
(b) the isolated protein of (a) which consists of an amino acid sequence selected from SEQ ID NOS: 14, 15, 16, 17, 18, 19, 20, 33, 34, 35, 36 and 37.

8. A composition comprising at least one of:
(a) an isolated nucleic acid molecule encoding the protein of claim 7,
(b) a vector comprising the nucleic acid molecule of (a), and
(c) an isolated cell comprising the vector of (b).

9. The method of claim 4 wherein the biological sample comprises a blood sample or a lavage sample.

10. The method of claim 9 wherein at least one of:
(a) heparin is present in the step of incubating,
(b) heparin and an added carbohydrate are present in the step of incubating, and
(c) at least one Mycobacterium protein, or a peptide fragment thereof or a chemical analog thereof, is present in the step of incubating, said Mycobacterium protein being selected from ESAT6, CFP10, TB7.7 and PPD.

11. The method of claim 9 wherein the Mycobacterium species is selected from M. tuberculosis, M. bovis, M. bovis BCG, M. africanum, M. canetti, M. caprae, M. microti, M. pinnipedii, M. avium, M. avium paratuberculosis, M. avium silvaticum, M. avium “hominissuis”, M. colombiense, M. asiaticum, M. gordonae, M. gastri, M. kansasii, M. hiberniae, M. nonchromogenicum, M. terrae, M. triviale, M. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. simiae, M. branderi, M. cookii, M. celatum, M. bohemicum, M. haemophilusalmoense, M. szulgai, M. leprae, M. lepraemurium, M. lepromatosis, M. africanum, M. botniense, M. chimaera, M. conspicuum, M. doricum, M. farcinogenes, M. heckeshornense, M. intracellulare, M. lacus, M. marinum, M. monacense, M. montefiorense, M. murale, M. nebraskense, M. saskatchewanense, M. scrofulaceum, M. shimoidei, M. tusciae, M. xenopi, M. intermedium, M. abscessus, M. chelonae, M. bolletii, M. fortuitum, M. fortuitum subsp. acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diernhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodii, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, M. seoulense and M. tokaiense.

12. The method of claim 9 wherein in the step of incubating, the at least one protein or peptide comprises an amino acid that is selected from SEQ ID NOS: 14, 15, 16, 17, 18, 19, 20, 33, 34, 35, 36 and 37.

13. The method of claim 9 wherein the effector molecule is selected from interferon-γ, TNF-α, and an interleukin.

14. The method of claim 9 wherein the effector molecule is interferon-γ.
